(19) United States
(12) Patent Application Publication
(10) Pub. No.: US 2012/0183973 A1
(43) Pub. Date: Jul. 19, 2012

(54) BIOMARKER COMPOSITION FOR DETECTING DIABETIC RETINOPATHY AND DIAGNOSTIC KIT THEREFOR

(75) Inventors: Young-Soo KIM, Seoul (KR); Hyeong-Gon YU, Seoul (KR); Kyung-Gon KIM, Seoul (KR); Sang-Jin KIM, Seoul (KR); Tae-Oh KIM, Yongin-si (KR)

(73) Assignee: SNU R & DB FOUNDATION, Seoul (KR)

(21) Appl. No.: 13/354,016

(22) Filed: Jan. 19, 2012

Related U.S. Application Data
(63) Continuation of application No. 12/733,330, filed on Feb. 24, 2010.

(30) Foreign Application Priority Data
Aug. 30, 2007 (KR) 10-2007-0087512

(51) Int. Cl. G01N 33/573; G01N 33/566

(52) U.S. Cl. 435/7.4; 436/501

(57) ABSTRACT

Provided is a biomarker composition for detecting diabetic retinopathy, comprising at least one protein selected from the group consisting of proteins as set forth in SEQ ID NOS: 1 to 169, a method and a kit for diagnosing diabetic retinopathy using the same. The biomarker can provide fundamental information in researching vitreoretinal disorders, such as, diabetic retinopathy and the protein may be used in a method and a kit for diagnosing diabetic retinopathy with a molecule specifically binding thereto.

### CROSS-REFERENCE TO PRIOR APPLICATIONS

This is a Continuation Application filed under 35 U.S.C. §120 as a continuation of U.S. patent application Ser. No. 12/733,330, filed on Feb. 24, 2010, which was a National Phase Application filed under 35 U.S.C. §371 as a national stage of PCT/KR2008/005046, filed on Aug. 28, 2008, an application claiming the benefit under 35 U.S.C. §119 of Korean Application No. 10-2007-0087512, filed on Aug. 30, 2007, the content of each of which is hereby incorporated by reference in their entirety.

### TECHNICAL FIELD

The present invention relates to a biomarker composition for detecting diabetic retinopathy; and a kit for diagnosing diabetic retinopathy. And also, the present invention relates to a biomarker composition for detecting diabetes mellitus; and a kit for diagnosing diabetes mellitus.

### REFERENCE TO THE INCORPORATED SEQUENCE LISTING IN TEXT

The Sequence Listing submitted in text format (.txt) on Jan. 19, 2012, named “Sequence_Listing.txt”, (created on Thursday, Jan. 19, 2011, 1.19 MB), is incorporated herein by reference.

### BACKGROUND ART

Diabetes mellitus comprises a group of metabolic disorder characterized by high blood glucose resulting from reduced insulin secretion, decreased glucose utilization, or increased glucose production. Moreover, at least 20 million people have diabetes in the United States [1]. Diabetes can lead to serious vascular complications, which include macrovascular complications like coronary heart disease, cerebrovascular disease, and peripheral vascular disease, and microvascular complications like diabetic retinopathy, nephropathy, and neuropathy.

Diabetic retinopathy (DR) occurs in three quarters of diabetics with a disease history of more than 15 years [2], and causes 12,000 to 24,000 new cases of blindness each year in the United States, which makes diabetes the leading cause of new cases of blindness among adults (20 to 74 years old) [1]. Pathologic changes in diabetic retinopathy include retinal vascular abnormalities, such as, the impairment of retinal blood flow, increased vascular permeability, breakdown of the blood-retinal barrier, and capillary occlusion resulting in localized hypoxia [3-6]. Moreover, as retinal hypoxia progresses, angiogenic factors are induced that promote retinal neovascularization.

Proliferative diabetic retinopathy (PDR) concerns new vessels growth into the vitreous cavity, and subsequent fibrovascular proliferation, retinal detachment, and vitreous hemorrhage in PDR, which eventually result in blindness. Although blindness rates have been reduced by panretinal laser photocoagulation and vitrectomy, the visual impairments caused by diabetic retinopathy remain of great concern [7, 8].

A number of studies have identified factors associated with the pathogenesis of PDR, e.g., angiogenic factors like vascular endothelial growth factor [9-12], angiotensin-converting enzyme [13], insulin-like growth factor [14], angiopoietin [15], erythropoietin [16], placenta growth factor [17], and advanced glycation end product [18], and anti-angiogenic factors like pigment epithelium derived factor [19-21]. However, the majority of previous studies have focused on sets of targeted proteins, particularly on the molecules involved in angiogenesis and cellular proliferation, which makes it difficult to evaluate changes in entire vitreous humor protein profiles and to identify novel markers of PDR pathogenesis.

Recent advances in two-dimensional gel electrophoresis (2-DE) and mass spectrometry (MS) have allowed the further exploration and acquisition of vitreous protein profiles [22-24]. In our previous study, by using both 2-DE and matrix-assisted laser desorption ionization-time of flight (MALDI-TOF) MS, we constructed PDR vitreous protein profiles and identified eight proteins that are possibly involved in the pathogenesis of PDR [25].

### PRIOR ART REFERENCES

[Reference 1] CDC, National Diabetes Fact Sheet: General information and National Estimates on Diabetes in the United States. US Department of Health and Human Services, Centers for Disease Control and Prevention, Atlanta, Ga. (2005).
[Reference 2] Klein, R., Klein, B. E., Moss, S. E., Cruickshanks, K. J., The Wisconsin Epidemiologic Study of Diabetic Retinopathy: XVII. The 14-year incidence and progression of diabetic retinopathy and associated risk factors in type 1 diabetes. Ophthalmology 1998, 105, 1801-1815.
[Reference 3] Schroder, S., Palinski, W., Schmid-Schonbein, G. W., Activated monocytes and granulocytes, capillary nonperfusion, and neovascularization in diabetic retinopathy. The American journal of pathology 1991, 139, 81-100.
[Reference 4] Krogsaa, B., Lund-Andersen, H., Mehlsen, J., Sestoft, L., Larsen, J., The blood-retinal barrier permeability in diabetic patients. Acta ophthalmologica 1981, 59, 689-694.
[Reference 5] Bursell, S. E., Clermont, A. C., Kinsley, B. T., Simonson, D. C., et al., Retinal blood flow changes in patients with insulin-dependent diabetes mellitus and no diabetic retinopathy. Investigative ophthalmology & visual science 1996, 37, 886-897.
[Reference 6] Gardner, T. W., Antonetti, D. A., Barber, A. J., LaNoue, K. F., Levison, S. W., Diabetic retinopathy: more than meets the eye. Survey of ophthalmology 2002, 47 Suppl 2, S253-262.
[Reference 7] Ferris, F. L., Davis, M., Early Treatment Diabetic Retinopathy Study Research Group. Early Treatment Diabetic Retinopathy Study Research Group No. 1: Photocoagulation for diabetic macular edema. Early treatment diabetic retinopathy study report no. 1: photocoagulation for diabetic macular edema. Arch. Ophthalmol. 1985, 103, 1796-1806.
[Reference 8] Lewis, H., Abrams, G. W., Blumenkranz, M. S., Campo, R. V., Vitrectomy for diabetic macular traction and edema associated with posterior hyaloidal traction. Ophthalmology 1992, 99, 753-759.
[Reference 9] Witmer, A. N., Blaauwgeers, H. G., Weich, H. A., Alitalo, K., et al., Altered expression patterns of VEGF receptors in human diabetic retina and in experimental VEGF-induced retinopathy in monkey. Investigative ophthalmology & visual science 2002, 43, 849-857.
[Reference 10] Pe'er, J., Folberg, R., Itin, A., Gnessin, H., et al., Upregulated expression of vascular endothelial growth factor in proliferative diabetic retinopathy. The British journal of ophthalmology 1996, 80, 241-245.
[Reference 11] Mathews, M. K., Merges, C., McLeod, D. S., Lutty, G. A., Vascular endothelial growth factor and vascular permeability changes in human diabetic retinopathy. Investigative ophthalmology & visual science 1997, 38, 2729-2741.
[Reference 12] Witmer, A. N., Vrensen, G. F., Van Noorden, C. J., Schlingemann, R. O., Vascular endothelial growth factors and angiogenesis in eye disease. Progress in retinal and eye research 2003, 22, 1-29.
[Reference 13] Kida, T., Ikeda, T., Nishimura, M., Sugiyama, T., et al., Renin-angiotensin system in proliferative diabetic retinopathy and its gene expression in cultured human muller cells. Japanese journal of ophthalmology 2003, 47, 36-41.
[Reference 14] Guidry, C., Feist, R., Morris, R., Hardwick, C. W., Changes in IGF activities in human diabetic vitreous. Diabetes 2004, 53, 2428-2435.
[Reference 15] Ohashi, H., Takagi, H., Koyama, S., Oh, H., et al., Alterations in expression of angiopoietins and the Tie-2 receptor in the retina of streptozotocin induced diabetic rats. Molecular vision 2004, 10, 608-617.
[Reference 16] Watanabe, D., K., S., Erythropoietin as a retinal angiogenic factor in proliferative diabetic retinopathy. The New England journal of medicine 2005, 353, 782-792.
[Reference 17] Mitamura, Y., Tashimo, A., Nakamura, Y., Tagawa, H., et al., Vitreous levels of placenta growth factor and vascular endothelial growth factor in patients with proliferative diabetic retinopathy. Diabetes care 2002, 25, 2352.
[Reference 18] Matsumoto, Y., Takahashi, M., Chikuda, M., Arai, K., Levels of mature cross-links and advanced glycation end product cross-links in human vitreous. Japanese journal of ophthalmology 2002, 46, 510-517.
[Reference 19] Dawson, D. W., Volpert, O. V., Gillis, P., Crawford, S. E., et al., Pigment epithelium-derived factor: a potent inhibitor of angiogenesis. Science (New York, N.Y. 1999, 285, 245-248.
[Reference 20] Duh, E. J., Yang, H. S., Suzuma, I., Miyagi, M., et al., Pigment epithelium-derived factor suppresses ischemia-induced retinal neovascularization and VEGF-induced migration and growth. Investigative ophthalmology & visual science 2002, 43, 821-829.
[Reference 21] Spranger, J., Osterhoff, M., Reimann, M., Mohlig, M., et al., Loss of the antiangiogenic pigment epithelium-derived factor in patients with angiogenic eye disease. Diabetes 2001, 50, 2641-2645.
[Reference 22] Nakanishi, T., Koyama, R., Ikeda, T., Shimizu, A., Catalogue of soluble proteins in the human vitreous humor: comparison between diabetic retinopathy and macular hole. Journal of chromatography 2002, 776, 89-100.
[Reference 23] Ouchi, M., West, K., Crabb, J. W., Kinoshita, S., Kamei, M., Proteomic analysis of vitreous from diabetic macular edema. Experimental eye research 2005, 81, 176-182.
[Reference 24] Yamane, K., Minamoto, A., Yamashita, H., Takamura, H., et al., Proteome analysis of human vitreous proteins. Mol Cell Proteomics 2003, 2, 1177-1187.
[Reference 25] Kim, S. J., Kim, S., Park, J., Lee, H. K., et al., Differential expression of vitreous proteins in proliferative diabetic retinopathy. Current eye research 2006, 31, 231-240.

### DETAILED DESCRIPTION OF THE INVENTION

### Technical Problem

In order to identify biomarkers capable of detecting PDR, the present inventors conducted extensive search on entire proteins involved in the pathogenesis of PDR, including low abundance proteins. As a result, 531 proteins were identified in the vitreous proteome and 240 proteins among them were newly identified. Among the newly identified 240 vitreous proteins, it was found that 105 proteins were significantly over-expressed in the vitreous humors obtained from PDR patients, while 64 proteins were significantly over-expressed in those obtained from normal people. And also, it has been found that the levels of thyroxine-binding globulin precursor (TBG) in both vitreous and plasma of PDR and NPDR states and in plasma of diabetes mellitus (DM) state, are outstandingly higher than in non-diabetic control (MH or normal control), which means that TBG can function as is a diabetes mellitus (DM) biomarker.

Thus, the present invention provides a biomarker composition for detecting diabetic retinopathy comprising one or more protein(s) among the differently expressed 169 proteins in the vitreous humors derived from PDR patients and normal people, respectively.

The present invention also provides a biomarker composition for detecting diabetes mellitus comprising thyroxine-binding globulin precursor, i.e., the protein as set forth in SEQ ID NO: 69.

The present invention also provides a kit for diagnosing diabetic retinopathy, comprising a molecule specifically binding to the protein(s).

The present invention also provides a kit for diagnosing diabetic mellitus, comprising a molecule specifically binding to thyroxine-binding globulin precursor, i.e., the protein as set forth in SEQ ID NO: 69.

### Technical Solution

According to an aspect of the present invention, there is provided a biomarker composition for detecting diabetic retinopathy comprising at least one protein selected from the group consisting of proteins as set forth in SEQ ID NOS: 1 to 169.

In the biomarker composition of the present invention, the at least one protein may be selected from the group consisting of proteins as set forth in SEQ ID NOS: 4, 5, 8, 15, 19, 27, 30, 32, 33, 36, 38, 39, 40, 41, 42, 45, 46, 47, 48, 49, 51, 52, 53, 55, 56, 59, 60, 62, 66, 67, 68, 69, 71, 74, 78, 83, 86, 88, 89, 91, 95, 96, 97, 98, 99, 100, and 105. And, the at least one protein may be selected from the group consisting of proteins as set forth in SEQ ID NOS: 109, 111, 117, 122, 123, 124, 125, 126, 127, 129, 131, 132, 136, 137, 138, 146, 147, 149, 152, 158, 159, 161, 165, and 167. Preferably, the at least one protein may be a protein as set forth in SEQ ID NOS: 48 or 69. And also, blood or urine may be used as a test sample.

According to another aspect of the present invention, there is provided a biomarker composition for detecting diabetes mellitus comprising the protein as set forth in SEQ ID NO: 69. In the biomarker composition, blood or urine may be used as a test sample.

According to still another aspect of the present invention, there is provided a kit for diagnosing diabetic retinopathy, comprising a molecule specifically binding to at least one protein selected from the group consisting of proteins as set forth in SEQ ID NOS: 1 to 169.

The molecule may be a monoclonal antibody, a polyclonal antibody, substrate, ligand, or cofactor. The at least one protein may be selected from the group consisting of proteins as set forth in SEQ ID NOS: 4, 5, 8, 15, 19, 27, 30, 32, 33, 36, 38, 39, 40, 41, 42, is 45, 46, 47, 48, 49, 51, 52, 53, 55, 56, 59, 60, 62, 66, 67, 68, 69, 71, 74, 78, 83, 86, 88, 89, 91, 95, 96, 97, 98, 99, 100, and 105. And, the at least one protein may be selected from the group consisting of proteins as set forth in SEQ ID NOS: 109, 111, 117, 122, 123, 124, 125, 126, 127, 129, 131, 132, 136, 137, 138, 146, 147, 149, 152, 158, 159, 161, 165, and 167. Preferably, the at least one protein may be a protein as set forth in SEQ ID NOS: 48 or 69. And also, in the kit of the present invention, blood or urine may be used as a test sample.

According to still another aspect of the present invention, there is provided a kit for diagnosing diabetes mellitus, comprising a molecule specifically binding to the protein as set forth in SEQ ID NO: 69. The molecule may be a monoclonal antibody, a polyclonal antibody, substrate, ligand, or cofactor; and blood or urine may be used as a test sample.

### ADVANTAGEOUS EFFECTS

By the present invention, it has been newly found that 105 proteins as set forth in SEQ ID NOS: 1 to 105 are significantly over-expressed in the vitreous humors obtained from PDR patients, while 64 proteins as set forth in SEQ ID NOS: 106 to 169 are significantly over-expressed in those obtained from normal people. Therefore, the proteins can be used for biomarker capable of detecting diabetic retinopathy. The biomarker can provide fundamental information in researching vitreoretinal disorders, such as diabetic retinopathy. Especially, the newly found proteins may be applied to a kit for diagnosing diabetic retinopathy with a molecule specifically binding thereto, e.g., a monoclonal antibody. And also, it has been newly found that the levels of thyroxine-binding globulin precursor (TBG) in both vitreous and plasma of PDR and NPDR states and in plasma of diabetes mellitus (DM) state, are outstandingly higher than in non-diabetic control (MH or normal control). Therefore, TBG may be applied to a kit for diagnosing diabetes mellitus with a molecule specifically binding thereto.

### BRIEF DESCRIPTION OF THE DRAWINGS

FIG. 1 shows scheme of the 2-DE/MALDI-MS, LC-MALDI-MS/MS, and LC-ESI-MS/MS experiments.

FIG. 2 shows Venn diagram of identified PDR proteins by 2-DE.

FIGS. 3 to 5 show process used to identify proteins by SDS-PAGE and LC-MALDI-MS/MS. Non-depleted PDR, albumin/IgG depleted PDR and control vitreous samples were separated by SDS-PAGE and their respective proteins were identified by LC-MALDI-MS/MS. In FIG. 3, 500 μg of non-depleted PDR vitreous was loaded on SDS-PAGE gel and sliced into 16 pieces. Each piece was chopped into fragments for in-gel digestion. In FIG. 4, in-gel digested tryptic peptides were injected into a nano LC system for fractionation. This LC chromatogram represents elution time (horizontal) versus peak intensity (vertical). LC chromatogram was generated according to the acetonitrile gradient over 60 min. In FIG. 5, spotted fractionated peptides on a 144 well MALDI-target plate were analyzed using a MALDI-TOF/TOF tandem spectrometer and the spectra of the 144 spots in the 9th SDS-PAGE gel slice were visualized using the peak explorer module of GPS explorer v3.5 (Matrix Science, Boston Mass.). The chart represents m/z (vertical) versus MALDI-target plate number (horizontal).

FIG. 6 shows MS/MS spectrum for the peptide LAAAVSNGFYDLYR (SEQ ID NO: 170), which originated from pigment epithelium-derived factor (PEDF), a representative protein in the 9th fraction of the SDS-PAGE gel. The chart represents m/z (horizontal) versus % intensity (vertical). The spectrums for the tryptic peptides of PEDF were annotated using GPS explorer software v3.5 and the MASCOT search engine v1.9 against IPI human database v3.24.

FIG. 7 shows Venn diagram of proteins identified by LC-MALDI-MS/MS and LC-ESI-MS/MS.

FIG. 8 shows subcategories under “biological process” of the GO annotation for three vitreous samples.

FIG. 9 shows the numbers of peptides for each PDR specific protein group. The larger the peptide number is, the easier to find the MRM transition.

FIG. 10 shows age distribution of the sample according to sex.

FIG. 11 shows the interactive plot and ROC curve of TBG, which is for MH (non-diabetic control) versus PDR in vitreous set.

FIG. 12 shows the interactive plots and the ROC curves of TBG for MH (non-diabetic control) versus NPDR vitreous set.

FIG. 13 shows the interactive plots of TBG for MH versus PDR in plasma sample set.

FIG. 14 shows the interactive plots and ROC curve of TBG for MH versus NPDR in plasma sample set.

FIGS. 15 and 16 show the levels of thyroxine-binding globulin precursor (TBG) of PDR and NPDR states in both vitreous (FIG. 15) and plasma (FIG. 16).

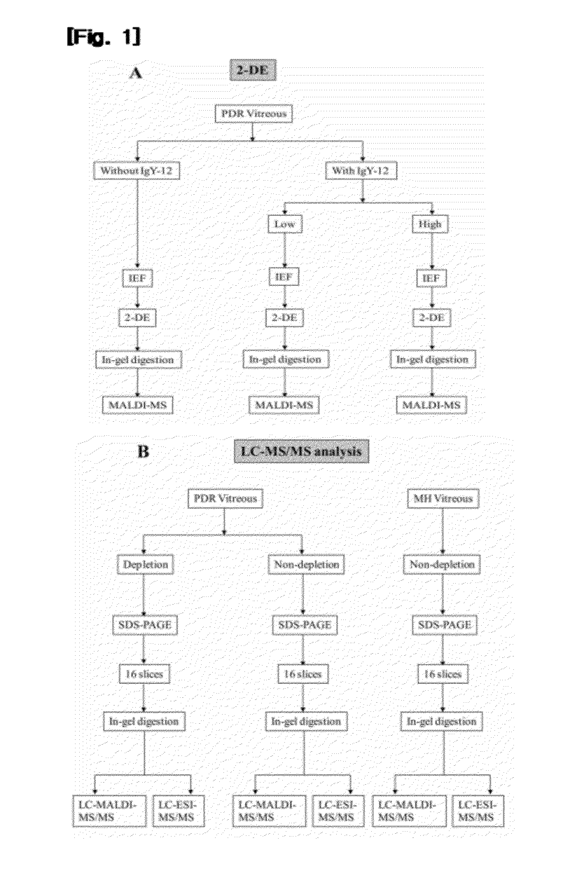

### BEST MODE FOR CARRYING OUT THE INVENTION

The present invention includes a biomarker composition for detecting diabetic retinopathy comprising at least one protein selected from the group consisting of proteins as set forth in SEQ ID NOS: 1 to 169.

The present inventors used several proteomic methods to identify components of the vitreous proteome, i.e., IS/2-DE/MALDI-MS, nano LC-MALDI-MS/MS, and nano LC-ESI-MS/MS. Proteins identified by nano LC-MALDI-MS/MS and nano LC-ESI-MS/MS were validated using the Trans-Proteomic Pipeline, in which isoforms and homologous proteins are grouped into representative orthologues. The present inventors also conducted LC-MS/MS analyses on albumin/IgG depleted PDR samples, non-albumin/IgG depleted PDR samples, and macular hole (MH) vitreous samples to conduct search of entire proteins involved in the pathogenesis of PDR, thereby identifying 531 proteins. As a result of database search on the 531 proteins, it was newly found that 240 proteins are involved in the PDR pathogenesis. Among them, it was found that 105 proteins described in Table 1 to 4 were significantly over-expressed in the vitreous humors obtained from PDR patients, while 64 proteins described in Table 5 to 6 were significantly over-expressed in those obtained from normal people.

TABLE 1

| | Detected | | | |
|---|---|---|---|---|
| | in | | IPI | |
| SEQ | plasma | | accession | Re- |
| ID | proteome | Protein name | number | marks |
| 1 | | 101 KDA PROTEIN | IPI00760855 | A |
| 2 | | 13 kDa protein | IPI00743473 | A |
| 3 | | 14-3-3 protein epsilon | IPI00000816 | A |
| 4 | * | 16 kDa protein | IPI00218733 | A |
| 5 | * | 184 KDA PROTEIN | IPI00303313 | A |
| 6 | | 57 kDa protein | IPI00383111 | A |
| 7 | | 97 KDA PROTEIN | IPI00794184 | A |
| 8 | * | Adiponectin precursor | IPI00020019 | A |
| 9 | | ADP-ribosylation factor 1 | IPI00215914 | A |
| 10 | | ALPHA3A | IPI00377045 | A |
| 11 | | ANNEXIN A2 ISOFORM 1 | IPI00418169 | A |
| 12 | | Beta-hexosaminidase | IPI00012585 | A |
| | | beta chain precursor | | |
| 13 | | Biglycan precursor | IPI00010790 | A |
| 14 | | Calcium/calmodulin- | IPI00005592 | A |
| | | dependent 3′,5′-cyclic | | |
| | | nucleotide phosphodiesterase 1B | | |
| 15 | * | CALMODULIN-LIKE PROTEIN 5 | IPI00021536 | A |
| 16 | | CD59 glycoprotein precursor | IPI00011302 | A |
| 17 | | CDNA FLJ25678 fis, clone | IPI00017672 | A |
| | | TST04067, highly | | |
| | | similar to PURINE NUCLEOSIDE | | |
| | | PHOSPHORYLASE | | |
| 18 | | CDNA FLJ41981 fis, clone | IPI00784830 | A |
| | | SMINT2011888, highly similar to | | |
| | | Protein Tro alphal H,myeloma | | |
| 19 | * | Cholinesterase precursor | IPI00025864 | A |
| 20 | | Cofilin-1 | IPI00012011 | A |
| 21 | | Corneodesmosin precursor | IPI00386809 | A |
| 22 | | Dermatopontin precursor | IPI00292130 | A |
| 23 | | E3 UBIQUITIN-PROTEIN | IPI00328911 | A |
| | | LIGASE HECTD1 | | |
| 24 | | Endothelial protein C | IPI00009276 | A |
| | | receptor precursor | | |
| 25 | | FERRITIN HEAVY CHAIN | IPI00554521 | A |
| 26 | | FERRITIN LIGHT | IPI00796538 | A |
| | | POLYPEPTIDE VARIANT | | |
| 27 | * | Fetuin-B precursor | IPI00005439 | A |
| 28 | | FIBRONECTIN 1 ISOFORM 4 | IPI00414283 | A |
| | | PREPROPROTEIN | | |
| 29 | | Fructose-bisphosphate aldolase C | IPI00418262 | A |
| 30 | * | Gamma-glutamyl hydrolase | IPI00023728 | A |
| | | precursor | | |

TABLE 2

| | Detected in | | | |
|---|---|---|---|---|
| SEQ | plasma | | IPI accession | |
| ID | proteome | Protein name | number | Remarks |
| 31 | | Gastrokine-1 precursor | IPI00021342 | A |
| 32 | * | Growth/differentiation factor 8 precursor | IPI00023751 | A |
| 33 | * | Hepatocyte growth factor activator precursor | IPI00029193 | A |
| 34 | | Hornerin | IPI00398625 | A |
| 35 | | Hypoxanthine-guanine phosphoribosyltransferase | IPI00218493 | A |
| 36 | * | Intercellular adhesion molecule 2 precursor | IPI00009477 | A |
| 37 | | Isoform 1 of Arginase-1 | IPI00291560 | A |
| 38 | * | Isoform 1 of Contactin-4 precursor | IPI00178854 | A |
| 39 | * | Isoform 1 of C-reactive protein precursor | IPI00022389 | A |
| 40 | * | Isoform 1 of Ficolin-3 precursor | IPI00293925 | A |
| 41 | * | Isoform 1 of Mannan-binding lectin serine | IPI00294713 | A |
| | | protease 2 precursor | | |
| 42 | * | Isoform 1 of Multiple epidermal growth factor-like domains 8 | IPI00027310 | A |
| 43 | | Isoform 1 of Phosphatidylinositol-glycan- | IPI00299503 | A |
| | | specific phospholipase D precursor | | |
| 44 | | ISOFORM 1 OF PHOSPHOLIPID TRANSFER | IPI00643034 | A |
| | | PROTEIN PRECURSOR | | |
| 45 | * | Isoform 1 of Plexin domain-containing protein 2 precursor | IPI00044369 | A |
| 46 | * | Isoform 1 of Probable helicase senataxin | IPI00142538 | A |
| 47 | * | Isoform A of Proteoglycan-4 precursor | IPI00024825 | A |
| 48 | * | Kallistatin precursor | IPI00328609 | A |
| 49 | * | Lipopolysaccharide-binding protein precursor | IPI00032311 | A |
| 50 | | Lithostathine 1 alpha precursor | IPI00009027 | A |
| 51 | * | Macrophage colony-stimulating factor 1 receptor precursor | IPI00011218 | A |
| 52 | * | MANIA1 PROTEIN | IPI00291641 | A |
| 53 | * | MIMECAN PRECURSOR | IPI00025465 | A |
| 54 | | MUCIN-5B PRECURSOR | IPI00384897 | A |
| 55 | * | Multimerin-2 precursor | IPI00015525 | A |
| 56 | * | Myocilin precursor | IPI00019190 | A |
| 57 | | Myoglobin | IPI00217493 | A |
| 58 | | Neurexin 3-alpha | IPI00216728 | A |
| 59 | * | Nidogen-2 precursor | IPI00028908 | A |
| 60 | * | PEPTIDYL-PROLYL CIS-TRANS ISOMERASE C | IPI00024129 | A |

TABLE 3

| | Detected in | | | |
|---|---|---|---|---|
| | plasma | | IPI accession | |
| SEQ ID | proteome | Protein name | number | Remarks |
| 61 | | Phosphatidylethanolamine-binding protein 1 | IPI00219446 | A |
| 62 | * | Pregnancy zone protein precursor | IPI00025426 | A |
| 63 | | Protein DJ-1 | IPI00298547 | A |
| 64 | | Pseudogene candidate | IPI00454869 | A |
| 65 | | Rho GDP-dissociation inhibitor 2 | IPI00003817 | A |
| 66 | * | Serpin B4 | IPI00010303 | A |
| 67 | * | SUPEROXIDE DISMUTASE [MN], | IPI00022314 | A |
| | | MITOCHONDRIAL PRECURSOR | | |
| 68 | * | Thioredoxin | IPI00216298 | A |
| 69 | * | Thyroxine-binding globulin precursor | IPI00292946 | A |
| 70 | | TRIOSEPHOSPHATE ISOMERASE 1 VARIANT | IPI00465028 | A |
| 71 | * | UNCHARACTERIZED PROTEIN C7ORF24 | IPI00031564 | A |
| 72 | | V1-17 protein | IPI00045547 | A |
| 73 | | V1-5 protein (Fragment) | IPI00553215 | A |
| 74 | * | von Willebrand factor precursor | IPI00023014 | A |
| 75 | | WSB-1 ISOFORM | IPI00383777 | A |
| 76 | | 10 kDa protein | IPI00740756 | C |
| 77 | | 25 kDa protein | IPI00448800 | C |
| 78 | * | 272 KDA PROTEIN | IPI00219299 | C |
| 79 | | 330 kDa protein | IPI00163866 | C |
| 80 | | 3′-5′ exoribonuclease CSL4 homolog | IPI00032823 | C |
| 81 | | ACF7 PROTEIN | IPI00183169 | C |
| 82 | | Actin, aortic smooth muscle | IPI00008603 | C |
| 83 | * | ATP-binding cassette, sub-family A, member 2 isoform a | IPI00307592 | C |
| 84 | | BONE MORPHOGENETIC PROTEIN | IPI00005731 | C |
| | | RECEPTOR TYPE IA PRECURSOR | | |
| 85 | | CDNA: FLJ21459 fis, clone COL04714 | IPI00001606 | C |
| 86 | * | CENTROMERE PROTEIN F | IPI00027157 | C |
| 87 | | CRYPTOCHROME-1 | IPI00002540 | C |
| 88 | * | Dpy-19-like protein 1 | IPI00007461 | C |
| 89 | * | EXOCYST COMPLEX COMPONENT 8 | IPI00028264 | C |
| 90 | | ISOFORM 1 OF ALANINE | IPI00152432 | C |
| | | AMINOTRANSFERASE 2 | | |

TABLE 4

| | Detected in | | | |
|---|---|---|---|---|
| | plasma | | IPI accession | |
| SEQ ID | proteome | Protein name | number | Remarks |
| 91 | * | ISOFORM 1 OF GRIP AND COILED-COIL | IPI00005631 | C |
| | | DOMAIN-CONTAINING PROTEIN 2 | | |
| 92 | | ISOFORM 1 OF PROBABLE E3 UBIQUITIN- | IPI00333067 | C |
| | | PROTEIN LIGASE HERC4 | | |
| 93 | | ISOFORM 1 OF | IPI00069084 | C |
| | | TRANSFORMATION/TRANSCRIPTION | | |
| | | DOMAIN-ASSOCIATED PROTEIN | | |
| 94 | | Isoform 1 of Uncharacterized protein C9orf84 | IPI00658203 | C |
| 95 | * | ISOFORM 2 OF CROSSOVER JUNCTION | IPI00073193 | C |
| | | ENDONUCLEASE EME1 | | |
| 96 | * | ISOFORM 4 OF NESPRIN-1 | IPI00247295 | C |
| 97 | * | Junctional adhesion molecule A precursor | IPI00001754 | C |
| 98 | * | Mucin 5 (Fragment) | IPI00103397 | C |
| 99 | * | POTASSIUM/SODIUM HYPERPOLARIZATION | IPI00031506 | C |
| | | ACTIVATED CYCLIC NUCLEOTIDE-GATED | | |
| | | CHANNEL 1 | | |
| 100 | * | PROTEIN BASSOON | IPI00020153 | C |
| 101 | | SIMILAR TO GENERAL TRANSCRIPTION | IPI00736974 | C |
| | | FACTOR II-I REPEAT DOMAIN-CONTAINING | | |
| | | PROTEIN 1 (GTF2I REPEAT DOMAIN- | | |
| | | CONTAINING PROTEIN 1) (MUSCLE TFII-I | | |
| | | REPEAT DOMAIN-CONTAINING PROTEIN 1) | | |
| | | (GENERAL TRANSCRIPTION FACTOR III) | | |
| | | (SLOW-MUSCLE-FIBER ENHANCER BINDING PRO | | |
| 102 | | Structural maintenance of chromosomes protein 1B | IPI00479260 | C |
| 103 | | Thyroid hormone receptor-associated protein 2 | IPI00400834 | C |
| 104 | | UNCHARACTERIZED PROTEIN C22ORF30 | IPI00643747 | C |
| 105 | * | Utrophin | IPI00009329 | C |

TABLE 5

| | Detected | | | |
|---|---|---|---|---|
| | in | | | |
| | plasma | | IPI accession | |
| SEQ ID | proteome | Protein name | number | Remarks |
| 106 | | 106 kDa protein | IPI00293088 | G |
| 107 | | 12 kDa protein | IPI00478441 | G |
| 108 | | 261 KDA PROTEIN | IPI00791343 | G |
| 109 | * | 31 KDA PROTEIN | IPI00166417 | G |
| 110 | | 53 kDa protein | IPI00020430 | G |
| 111 | * | 72 kDa type IV collagenase precursor | IPI00027780 | G |
| 112 | | Agrin precursor | IPI00374563 | G |
| 113 | | Alcadein beta | IPI00396423 | G |
| 114 | | Alpha-mannosidase 2 | IPI00003802 | G |
| 115 | | Alpha-N-acetylgalactosaminidase precursor | IPI00414909 | G |
| 116 | | Beta-1,3-N-acetylglucosaminyltransferase radical | IPI00001793 | G |
| | | fringe | | |
| 117 | * | Caspase-14 precursor | IPI00013885 | G |
| 118 | | CDNA FLJ45402 fis, clone BRHIP3029409, | IPI00384783 | G |
| | | moderately similar to Homo sapiens secreted | | |
| | | frizzled-related protein 1 | | |
| 119 | | Chromogranin A precursor | IPI00290315 | G |
| 120 | | Deoxyribonuclease-2-alpha precursor | IPI00010348 | G |
| 121 | | DIS3 MITOTIC CONTROL HOMOLOG (S. | IPI00291003 | G |
| | | CEREVISIAE)-LIKE | | |
| 122 | * | EXTL2 protein (Fragment) | IPI00002732 | G |
| 123 | * | Extracellular matrix protein 1 precursor | IPI00003351 | G |
| 124 | * | Full-length cDNA clone CSODLOO4YM19 of B | IPI00328493 | G |
| | | cells (Ramos cell line) of Homo sapiens (Fragment) | | |
| 125 | * | Glucosidase 2 subunit beta precursor | IPI00026154 | G |
| 126 | * | Glutaminyl-peptide cyclotransferase precursor | IPI00003919 | G |
| 127 | * | Histatin-1 precursor | IPI00012024 | G |
| 128 | | Histone H4 | IPI00453473 | G |
| 129 | * | Isoform 1 of Contactin-associated protein-like 2 precursor | IPI00029343 | G |
| 130 | | Isoform 1 of Follistatin-related protein 4 precursor | IPI00477747 | G |
| 131 | * | Isoform 1 of L-lactate dehydrogenase A chain | IPI00217966 | G |
| 132 | * | Isoform 1 of Neogenin precursor | IPI00023814 | G |
| 133 | | Isoform 1 of Neural cell adhesion molecule L1 precursor | IPI00027087 | G |
| 134 | | Isoform 1 of Neurexin-2-alpha precursor | IPI00007921 | G |
| 135 | | Isoform 1 of Peptidyl-glycine alpha-amidating | IPI00177543 | G |
| | | monooxygenase precursor | | |

TABLE 6

| | Detected in | | | |
|---|---|---|---|---|
| | plasma | | IPI accession | |
| SEQ ID | proteome | Protein name | number | Remarks |
| 136 | * | Isoform 1 of Receptor-type tyrosine-protein | IPI00011642 | G |
| | | phosphatase delta precursor | | |
| 137 | * | Isoform 1 of Sulfhydryl oxidase 1 precursor | IPI00003590 | G |
| 138 | * | Isoform 1 of Tenascin-R precursor | IPI00160552 | G |
| 139 | | Isoform 2 of Neurexin-3-alpha precursor | IPI00441515 | G |
| 140 | | Isoform 2 of Phospholipid transfer protein precursor | IPI00217778 | G |
| 141 | | Isoform 2 of Testican-3 precursor | IPI00419590 | G |
| 142 | | Isoform 2 of Triosephosphate isomerase | IPI00451401 | G |
| 143 | | Isoform 4 of Seizure 6-like protein precursor | IPI00157417 | G |
| 144 | | Isoform Long of Alpha-mannosidase IIx | IPI00027703 | G |
| 145 | | Isoform Long of Iduronate 2-sulfatase precursor | IPI00026104 | G |
| 146 | * | Isoform Sap-mu-0 of Proactivator polypeptide precursor | IPI00012503 | G |
| 147 | * | ISOFORM XB OF TENASCIN-X PRECURSOR | IPI00025276 | G |
| 148 | | Laminin subunit beta-2 precursor | IPI00296922 | G |
| 149 | * | Laminin subunit gamma-1 precursor | IPI00298281 | G |
| 150 | | Latent-transforming growth factor beta-binding | IPI00292150 | G |
| | | protein 2 precursor | | |
| 151 | | Legumain precursor | IPI00293303 | G |
| 152 | * | L-lactate dehydrogenase B chain | IPI00219217 | G |
| 153 | | Lysosomal protective protein precursor | IPI00021794 | G |
| 154 | | Malate dehydrogenase, cytoplasmic | IPI00291005 | G |
| 155 | | N-acetylglucosamine-6-sulfatase precursor | IPI00012102 | G |
| 156 | | Neurocan core protein precursor | IPI00159927 | G |
| 157 | | Neuronal pentraxin-2 precursor | IPI00026946 | G |
| 158 | * | Oligodendrocyte-myelin glycoprotein precursor | IPI00295832 | G |
| 159 | * | Protein S100-A9 | IPI00027462 | G |
| 160 | | retbindin | IPI00027765 | G |
| 161 | * | Retinoic acid receptor responder protein 2 precursor | IPI00019176 | G |
| 162 | | Secreted frizzled-related protein 2 precursor | IPI00027596 | G |
| 163 | | Secreted frizzled-related protein 3 precursor | IPI00294650 | G |
| 164 | | similar to 60S ribosomal protein L23a | IPI00001310 | G |
| 165 | * | TBC1 domain family member 1 | IPI00164610 | G |
| 166 | | Testican-1 precursor | IPI00005292 | G |
| 167 | * | transmembrane protein 132A isoform b | IPI00301865 | G |
| 168 | | Two-pore calcium channel protein 2 | IPI00169371 | G |
| 169 | | V2-7 PROTEIN | IPI00747752 | G |

*: Detected in plasma proteome
Remark :
A-Expressed only in albumin/IgG depleted-PDR
B-Expressed in both albumin/IgG depleted-PDR and non-albumin/IgG depleted-PDR
C-Expressed only in non-albumin/IgG depleted-PDR
G-Expressed only in control vitreous humor.

As used herein, the term “at least one protein selected from the group consisting of proteins as set forth in SEQ ID NOS: 1 to 169” refers to protein(s) having one or more amino acid sequence(s) selected among the amino acid sequences as set forth in SEQ ID NOS: 1 to 169. It should be noted that the term “protein(s)”, as used herein, includes both each amino acid sequence of SEQ ID NOS: 1 to 169 and its fragments.

The biomarker composition of the present invention may be used for detecting proteins as set forth in SEQ ID NOS: 1 to 169 in a test sample, e.g., human tissue or humor. Especially, when human blood or urine is used as a test sample, potential ethical problems can be avoided. Thus, preferably, the biomarker composition of the present invention comprises protein(s) specifically over-expressed in the plasma as well as the vitreous humor. That is, preferably, the biomarker composition for detecting PDR of the present invention comprises protein(s) specifically over-expressed in the plasma, i.e., at least one protein selected from the group consisting of proteins as set forth in SEQ ID NOS: 4, 5, 8, 15, 19, 27, 30, 32, 33, 36, 38, 39, 40, 41, 42, 45, 46, 47, 48, 49, 51, 52, 53, 55, 56, 59, 60, 62, 66, 67, 68, 69, 71, 74, 78, 83, 86, 88, 89, 91, 95, 96, 97, 98, 99, 100, and 105; or at least one protein selected from the group consisting of proteins as set forth in SEQ ID NOS: 109, 111, 117, 122, 123, 124, 125, 126, 127, 129, 131, 132, 136, 137, 138, 146, 147, 149, 152, 158, 159, 161, 165, and 167. Preferably, the at least one protein is a protein as set forth in SEQ ID NOS: 48 or 69.

In the biomarker composition of the present invention, detection of the biomarker may be carried out by directly detecting the presence of a biomarker protein through two-dimensional gel electrophoresis (2-DE) on a test sample, e.g., human tissue or humor; or by indirectly identifying the presence of a biomarker protein through immunoassay methods using antigen-antibody reaction after contacting a test sample, e.g., human tissue or humor, with an antibody. The immunoassay methods include enzyme-linked immunoassay (ELISA, coated tube), immunomagnetic assay using antibody-linked magnetic beads, latex-bead assay method using antibody-linked latex beads.

And also, it has been found that the levels of thyroxine-binding globulin precursor (TBG) in both vitreous and plasma of PDR and NPDR states and in plasma of diabetes mellitus (DM) state, are outstandingly higher than in non-diabetic control (MH or normal control), which means that TBG can function as a diabetes mellitus (DM) biomarker. Therefore, the present invention includes a biomarker composition for detecting diabetes mellitus comprising the protein as set forth in SEQ ID NO: 69. In the biomarker composition, blood or urine may be used as a test sample.

The present invention includes a kit for diagnosing diabetic retinopathy, comprising a molecule specifically binding to at least one protein selected from the group consisting of proteins as set forth in SEQ ID NOS: 1 to 169.

The molecules may be a monoclonal antibody, a polyclonal antibody, substrate, ligand, or cofactor, which specifically binds to the at least one protein, preferably a monoclonal antibody or a polyclonal antibody, more preferably a monoclonal antibody.

Polyclonal or monoclonal antibodies may be prepared by a method commonly known is in the biotechnology field, e.g., hybridoma methods, such as those described by Kohler and Milstein, Nature, 256:495 (1975), and improvements thereto. For example, a mouse is immunized with a protein selected from the proteins having amino acid sequences as set forth in SEQ ID NOS: 1 to 169 or its fragment; or immunized with a synthetic peptide thereof bound to bovine serum albumin. Antigen-producing B lymphocytes isolated from the mouse are fused with human or mouse myeloma to produce immortalized hybridoma cell lines. The production of monoclonal antibodies is confirmed, e.g., through indirect ELISA methods, and then positive clones are selected. The positive clones are cultured and purified to obtain monoclonal antibodies, or alternatively, monoclonal antibodies are obtained by injecting the positive clones into mouse abdominal cavity and then taking the ascites.

As mentioned above, when human blood or urine is used as a test sample, potential ethical problems can be avoided. Thus, preferably, the kit of the present invention comprises a molecule specifically binding to at least one protein specifically over-expressed in the plasma as well as the vitreous humor, which may be selected from the group consisting of proteins as set forth in SEQ ID NOS: 4, 5, 8, 15, 19, 27, 30, 32, 33, 36, 38, 39, 40, 41, 42, 45, 46, 47, 48, 49, 51, 52, 53, 55, 56, 59, 60, 62, 66, 67, 68, 69, 71, 74, 78, 83, 86, 88, 89, 91, 95, 96, 97, 98, 99, 100, and 105; or selected from the group consisting of proteins as set forth in SEQ ID NOS: 109, 111, 117, 122, 123, 124, 125, 126, 127, 129, 131, 132, 136, 137, 138, 146, 147, 149, 152, 158, 159, 161, 165, and 167. Preferably, the at least one protein is a protein as set forth in SEQ ID NOS: 48 or 69.

And also, in the kit of the present invention, blood or urine may be preferably used as a test sample.

As mentioned above, it has been found that the levels of thyroxine-binding globulin precursor (TBG) in both vitreous and plasma of PDR and NPDR states and in plasma of diabetes mellitus (DM) state, are outstandingly higher than in non-diabetic control (MH or normal control), which means that TBG can function as a diabetes mellitus (DM) biomarker. Therefore, the present invention includes a biomarker composition for detecting diabetes mellitus comprising the protein as set forth in SEQ ID NO: 69. The molecule may be a monoclonal antibody, a polyclonal antibody, substrate, ligand, or cofactor; and blood or urine may be used as a test sample.

Hereinafter, the present invention will be described more specifically with reference to the following examples. The following examples are only for illustrative purposes and are not intended to limit the scope of the invention.

### Example 1

### 1. Test Method

(1) Patients and Vitreous Collection

We collected undiluted vitreous samples from 8 eyes of 8 PDR patients for the 2-DE experiment and from 11 eyes of 11 PDR patients for LC-MS/MS, during operations for tractional retinal detachment involving the macular region. Only patients that exhibited active neovascular membranes in extensive retinal areas were included, and those with gross vitreous hemorrhage or a history of recent vitreous hemorrhage, previous ocular surgery (including cataract surgery), or of another ocular disease, such as uveitis, were excluded. In order to acquire control samples from non-diabetic patients, we collected vitreous samples from 14 eyes with a small idiopathic macular hole (MH) (see Table 7).

TABLE 7

| Sample set | Mean age | Mean concentration: μg/μl |
|---|---|---|
| (patient numbers) | (range) | (range) |
| PDR for 2-DE | 62.5 | 5.6 |
| (n = 8) | (37-72) | (3.3-7.5) |
| PDR for LC-MS/MS | 56.0 | 6.4 |
| (n = 11) | (52-73) | (2.6-9.7) |
| MH for LC-MS/MS | 63.0 | 0.43 |
| (n = 14) | (45-71) | (0.10-1.21) |

MH vitreous samples were considered as non-diabetic controls because MH appears to develop as the result of vitreofoveal traction. Patients with other ocular diseases attributed to minor pathologic conditions were also excluded. All patients provided informed consent before being enrolled in the study, in accord with the protocol approved by the Institutional Review Board at Seoul National University Hospital. All protocols used in this study were also in full accord with the tenets of the Declaration of Helsinki.

Undiluted vitreous samples (0.5-0.8 ml) were collected at the commencement of pars plana vitrectomies performed using a Millennium microsurgical system (Bausch & Lomb, Rochester, N.Y.). In order to maintain intraocular pressure, vitreous was removed slowly with a vitreous cutter connected to a 1.0 ml syringe, while the sclera was indented. Harvested vitreous samples were collected in tubes, placed immediately on ice, and stored at −70° C. until required.

(2) Vitreous Sample Preparation

PDR and MH control samples were filtered/centrifuged at 15,000 g using 0.22 μm GV DURAPORE filter (Millipore company, Carrigtwohill, Co. Cork, Ireland) until all sample loaded passed completely through the filter. Protein concentrations were then determined using Bio-Rad protein assay reagents (Bio-Rad Laboratories, Hercules, Calif.). Generally, the protein concentrations of PDR samples were higher than those of controls (ca., 10 times higher; PDR samples 2.0˜10.0 μg/μl, control samples 0.1˜1.2 μg/μl). After collecting these clarified (filtered/centrifuged) vitreous samples from PDR and MH patients, 500 μl of individual samples from PDR or control MH patients were respectively pooled for 2-DE and LC-MS/MS experiments.

(3) Two Dimensional Gel Electrophoresis of Non-IS-Depleted PDR Samples

About 560 μg proteins in 100 μl of pooled PDR vitreous samples were subjected to TCA/acetone precipitation. Five volumes of 10% TCA in acetone containing 20 mM DTT was added to vitreous solution, stored at −20° C. for 4 hours, centrifuged at 28,000 g for 10 min, and the supernatant was then discarded. Five volumes of ice-cold acetone were added to the precipitant and the supernatant was then discarded to remove remaining TCA. After drying the pellet obtained using a speed vacuum, they were suspended in 250 μl rehydration buffer [7 M urea, 2 M thiourea, 2% CHAPS, 60 mM DTT and 0.5% (v/v) pharmalyte (pH 3-10)]. The concentration of pelleted vitreous protein in the rehydration solution was about 2 μg/μl, a calculated loss of ca. 25%. Pre-cast immobilized pH gradient strips (IPG strips, 13 cm, pH 4-7, linear, Amersham Biosciences, Uppsala, Sweden) were rehydrated overnight (12 hr) in a cassette using rehydration buffer. After aligning an IPG strip on an IEF tray, the voltage was increased incrementally. Initially, 500 V was applied for 1 hr, then 1000 V for 1 hr, and finally, 8000 V was applied to 14,500 VHr. IPG strips were equilibrated for 30 min in reducing solution (50 mM TrisHCl, pH 8.8, 6 M urea, 30% (v/v) glycerol, 2% (w/v) sodium dodecyl sulfate, 1% (w/v) DTT), and then for 30 min in the alkylating solution (identical to the reducing solution except that 2.5% (w/v) iodoacetamide was substituted for DTT). SDS-PAGE was conducted using 10% polyacrylamide gel using a standard SDS-PAGE protocol and an SE 600 Ruby gel unit (GE Healthcare, Uppsala, Sweden). Gels obtained were stained with silver staining solution. Three individual 2-DE experiments were carried out to obtain consistently detected spots.

(4) Two Dimensional Gel Electrophoresis of is-Depleted PDR Samples

The 12 high abundant proteins were depleted from PDR vitreous samples using an immunoaffinity subtraction (IS) system (Beckman Coulter ProteomeLab IgY-12 column, Beckman Coulter, Fullerton, Calif.), according to the manufacturer's instructions. This unit depleted the following 12 proteins: human serum albumin, IgG, fibrinogen, transferrin, IgA, IgM, HDL (apo A-I, apo A-II), haptoglobin, α1-antitrypsin, α1-acid glycoprotein, and α2-macroglobulin. 600 μg of PDR vitreous proteins were loaded on the IgY-12 column six times for column capacity reasons. Low abundance proteins were obtained in the flow-through fraction, whereas high abundance proteins bound to the antibody resin, and were recovered using stripping buffer, according to the manufacturer's instructions. Peptides in the flow-through and bound fractions were desalted by dialysis using Slide-A-Lyzer 3.5K dialysis cassettes kits (PIERCE, Rockford, Ill.) against 2 liters of distilled water three times. Thereafter, buffer exchange was carried out using an Amicon Ultra-4 10,000 (MILLIPORE, Bedford, Mass.) using 5 ml of rehydration buffer. The two resulting desalted samples (low and high abundance proteins) were then separated and visualized by 2-DE, respectively, as described in the previous section. Three individual 2-DE experiments were carried out to obtain consistently detected spots.

(5) In-Gel Trypsin Digestion

Excised gel pieces were destained in 30 mM potassium ferricyanide/100 mM sodium thiosulfate and then rinsed several times with 150 μl of distilled water until the yellow color of the ferricyanide completely disappeared. They were then dehydrated in 100% acetonitrile until they turned opaque white and rehydrated with 100 mM ammonium bicarbonate until transparent. This dehydration and rehydration process was repeated three times, and was followed by a single dehydration in 100% acetonitrile. The gel pieces were then dried in a vacuum centrifuge and rehydrated at 4° C. for 45 min in digestion buffer containing modified porcine trypsin in 50 mM ammonium bicarbonate at a concentration of 0.01 μg/μl (Promega, Madison, Wis.). Excess supernatant was then removed and gel pieces were soaked in 30 μl of 50 mM ammonium bicarbonate (NH₄HCO₃) overnight (16 hr) at 37° C. The solutions, which then contained cleaved peptides, were moved to new tubes.

(6) Peptide Mass Fingerprinting for 2-DE

Self-pack poros 20 R2 (Applied Biosystems, Foster City, Calif.) resin was packed inside a GEloader tip (Eppendorf AG, Hamburg, Germany), the end of which was twisted to cause the packed resin reside to be ca. 2 mm long. The trypsin-digested peptides were bound to the resin and washed with 0.1% Trifluoroacetic Acid (TFA). Bound peptides were eluted with 1 μl of sample matrix (5 mg/ml of α-cyano-4-hydroxy cinnamic acid in 70% ACN and 0.1% TFA solution). Eluted peptides were spotted on a 196 well MALDI plate. A 4700 proteomics analyzer (Applied Biosystems, Foster City, Calif.) was used in MS mode to identify proteins by peptide mass fingerprinting (PMF). The instrument was calibrated using 4700 cal mix (Applied Biosystems, Foster City, Calif.), which contained des-Arg-Bradykinin (monoisotopic mass: 904.4681), angiotensin I (monoisotopic mass: 1296.6853), Glu-Fibrinopeptide B (monoisotopic mass: 1570.6774), ACTH (1-17 clip, monoisotopic mass: 2093.0867), ACTH (18-39 clip, monoisotopic mass: 2465.1989) and ACTH (7-38 clip, monoisotopic mass: 3657.9294). MS data were acquired using 3,000 shots of a fixed intensity Nd:YAG laser at 355 nm and 200 Hz.

(7) PMF Data Analysis for 2-DE

The PMF proteomic search for in-gel digested peptide sample from 2-DE was conducted using GPS explorer software v3.5 and MASCOT v1.9 (Matrix Science, Boston, Mass.) as the database search engine. The minimum S/N was set at 10 and the following contaminant peaks were excluded during the search: 842.4, 870.5, 856.5, 771.1, 1794.8, 1475.7, 1993.9, 1383.6, 2211.1, 2705.1, 3338.8, 886.9, 893.0. The maximum number of missed cleavages was set to 1 for trypsin as protease and the precursor charge at +1. The differential peptide modifications allowed were the carbamidomethylation of cysteines and the oxidation of methionines. Acquired mass values were searched against the NCBInr database (updated 20 Feb., 2007) with a peptide mass tolerance of 150 ppm. Only identified proteins with a Confidence Index (C.I.)>95% were accepted.

(8) Nano LC Separation and Protein Identification by LC-MALDI-MS/MS Analysis

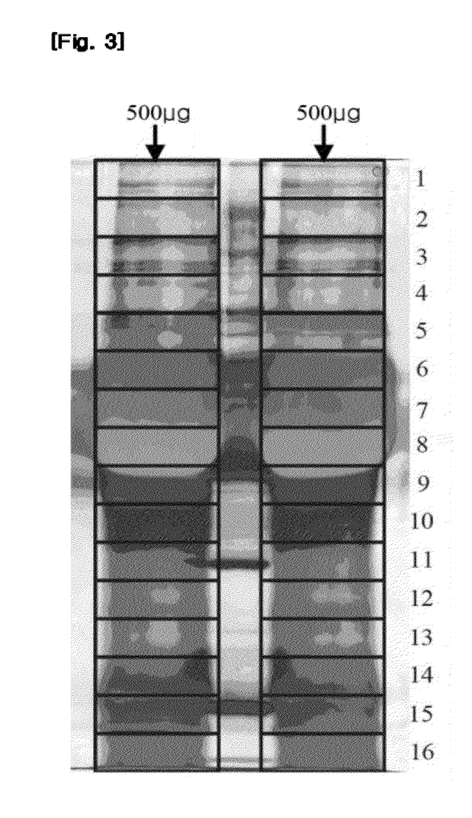

Albumin/IgG depleted PDR samples from 11 PDR patients, non-Albumin/IgG-depleted PDR samples from the same 11 patients, and control samples from 14 MH patients (Table 16) were pooled and loaded on SDS-PAGE gel (10% gel). One mg of each sample set (albumin/IgG depleted PDR, non-depleted PDR and non-depleted control) were loaded on two lanes (500 μg on each lane, FIG. 3A). The albumin/IgG depleted PDR samples were prepared using a ProteoExtract albumin/IgG removal kit (Calbiochem, San Diego, Calif.) to deplete albumin and IgG in PDR samples before loading them onto SDS-PAGE. After silver staining, gels were sliced into 16 pieces, and each piece was subjected to in-gel digestion as described above. The digested peptides were the vacuum-dried and resolved in 0.1% TFA or 0.1% formic acid in water. They were then desalted and concentrated using Ziptip_{C18} Pipette Tip (Millipore, Mass.).

The nano LC system used was an Ultimate 3000 unit (Switchos and Probot, Dionex, Amsterdam) coupled off-line to a MALDI-TOF/TOF (off-line LC-MALDI-MS/MS). This system was equipped with μ-Precolumn Cartridge (300 um i.d.×5 mm, C18 pepmap100, 5 μm, 100° C., Dionex, Amsterdam) and a reverse phase nano series column (75 μm i.d.×15 cm long column, C18 PepMap100, 3 μm, 100° C., Dionex). Initially, the trypsin generated peptide fragments were dissolved in 20 μl of 0.1% TFA and injected into the nano LC system using an autosampler equipped with a 20 μl sample loop. Injection was conducted in partial loop mode using a 10 μl injection volume. The trypsin generated peptide fragments were initially trapped in a precolumn, which was then washed with 0.05% TFA at 0.030 ml/min for 5 min. The precolumn containing bound peptides was then connected to 15 cm nano column using a valve switch.

The mobile phase to elute the peptide fragments consisted of 0.05% TFA, 5% acetonitrile in water (solution A) and 0.04% TFA, 80% acetonitrile in water (Solution B). Exponential gradient elution was performed by increasing the mobile phase composition from 0 to 50% of solution B over 30 min. The gradient was then ramped to 90% B for 5 min and back to 0% solution B for 20 min to equilibrate the column for the next run. The total run time was 60 min. This gradient was applied to the nano column at 300 nl/min at room temperature. Eluent was monitored at 214 nm by UV absorbance. Fractionated peptides were spotted on a 144 well MALDI plate at 20 sec per spot using the Probot system (Dionex). The matrix solution (6.2 mg/ml of α-cyano-4-hydroxy cinnamic acid (Agilent Technologies, Santa Clara, Calif.) in 36.0% methanol, 56.0% acetonitrile and 8.0% distilled water) was mixed with the mobile phase at 0.976 μl/min when spotting on the MALDI plate.

Peptide mass values were analyzed using the parameters mentioned for 2-DE analysis above and the 4700 analyzer. The 15 most intense peptides with S/N ratios exceeding 10 were subjected to MS/MS. The collision energy was set at 1 kV and the collision gas was air. MS/MS analysis was conducted using GPS explorer software (v3.5) and the MASCOT search engine (v1.9) using the same parameters used for 2-DE PMF analysis, but without exclusion peak filtering. Searching was performed against the Human International Protein Index (IPI) protein sequence database and included searches for known contaminants (IPI versions 3.24). The MASCOT search result from LC-MALDI-MS/MS analysis with the ‘dat’ file extension, was converted to pepXML file for further validation using the Trans-Proteomic Pipeline (TPP), according to instructions on the web.

(9) Nano LC Separation and Protein Identification by LC-ESI-MS/MS

In contrast with the LC-MALDI-MS/MS method which is based on MALDI ionization and the MASCOT algorithm, LC-ESI-MSMS results were based on ESI ionization and the SEQUEST algorithm. Thus, the other half of in-gel digested peptide samples from SDS-PAGE gel were used for protein identification using nano LC-ESI-MS/MS.

A binary Agilent nanoflow 1200 series HPLC system (Agilent Technologies Inc., is Santa Clara, Calif.) was directly coupled to a Thermo Electron model LTQ electrospray ionization linear single-quadrupole ion trap mass spectrometer (Thermo Fisher Scientific, Inc. Waltham, Mass.) fitted with an automatic gain control to avoid space charge limitations. In-gel digested peptides in 10 μl of aqueous formic acid (0.1%) were injected into the nano LC-ESI-MS/MS instrument. Peptides were separated by reverse-phase column chromatography and loaded on a 12 cm×75 μm capillary column packed in-house (Magic C18aq, Michrom Bioresources, Inc., Auburn, Calif.) using helium pressure cells. Gradient elution of the proteome sample was achieved using 90% solvent A (0.1% formic acid in H₂O) to 40% solvent B (0.1% formic acid in acetonitrile) at 250 nl/min over 120 min. A blank run was performed between sample runs to avoid cross contamination.

We used MS survey scanning from 300-2000 m/z followed by three data-dependent MS/MS scans (isolation width 2 m/z, normalized collision energy 35%, dynamic exclusion duration 30 s). Protein identifications from tandem mass spectra were first carried out using SEQUEST search software (Sequest cluster v3.2, initial mass tolerances for protein identification from MS peaks was 3 Da, and from MS/MS peaks was 1 Da. Two missed cleavages were allowed.) against the same IPI database as the MASCOT search mentioned above. SEQUEST search results based on LC-ESI-MS/MS analysis (LTQ) were converted to pepXML file for further validation using TPP.

(10) Filtering Search Results Using the Trans-Proteomic Pipeline

Search result files from MASCOT and SEQUEST in pepXML format were processed using the PeptideProphet and ProteinProphet modules in TPP, according to the instructions given. Peptides sequenced by MS/MS analysis were validated by PeptideProphet such that all sequenced peptides were allocated a probability based on parameters, such as, ion score, identity score, homology score, NTT in the case of MASCOT results, and Xcorr, dCn, Sp, NTT for SEQUEST results. ProteinProphet validated these peptides and determined the proteins most likely to contain these peptides. Probability cut-offs for running the PeptideProphet and ProteinProphet modules were set at 0.50 and 0.90, respectively. All processes like creating pepXML and determining scoring probabilities by PeptideProphet and ProteinProphet were carried out against the MASCOT and SEQUEST database mentioned above. Final TPP outputs from ProteinProphet were exported to Excel files for data merging and comparison. Processing by TPP allowed us to determine definite vitreous proteome profiles that consisted of proteins with high probability and reduced redundancy in the protein lists.

(11) Processing for Gene Ontology Annotation

IPI accession numbers were translated into Uniprot accession numbers (Swiss-prot numbers or TrEMBL numbers) by manually looking at matched accession numbers in the IPI database. Gene ontology (GO) was then assigned to Uniprot numbers using the QuickGO web tool. Each Uniprot number was assigned to three categories, i.e., biological process, function, and component. To avoid complexities resulting from detailed GO annotation, GO slim (level 3) was applied. If a single protein had been annotated by several processes, functions or components, all of such annotations were reflected in data representation redundantly.

### 2. Results and Discussion

(1) Protein Identification from PDR Vitreous Humor by Two-Dimensional Gel Electrophoresis

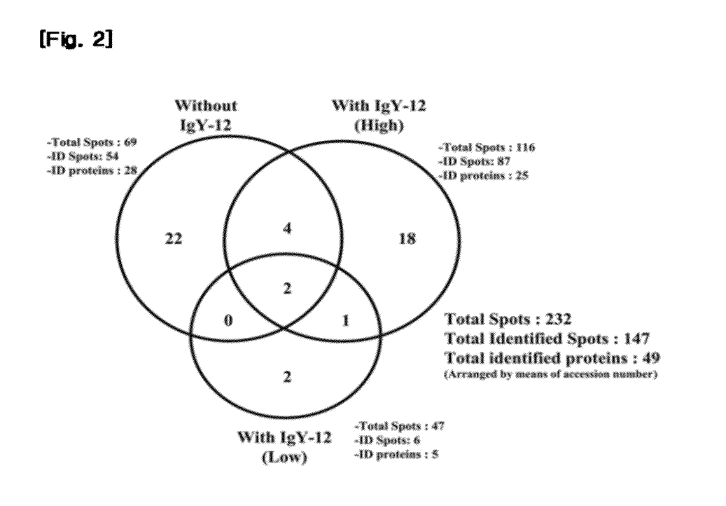

IgY-12 columns have been previously used to deplete 12 highly abundant proteins from human or primate biological fluids. Likewise, PDR vitreous samples were treated using IgY-12 columns, and subsequently the high and low abundance protein fractions obtained were subjected to 2-DE. Forty-seven spots were excised from the low abundance protein gel and 6 spots were matched to the NCBInr database (12.8%) and 5 proteins were identified (see FIG. 2). 116 spots were excised from the high abundance protein gel and 87 were matched to the database (75.0%) and 25 proteins were identified (see FIG. 2). In addition, we performed 2-DE on PDR samples not subjected to immunoaffinity subtraction (IS). In total 69 spots were excised, 54 were matched (78.3%), and 28 proteins were identified (see FIG. 2). From the identified protein lists for all three samples, 49 proteins were identified (see FIG. 2).

The identification rate was low in the low abundance protein gel. Of the 47 picked spots, only 6 were matched to the NCBInr database (12.8%). This may have been due to the low concentration of spots after in-gel digestion or the low yields of low abundance proteins. Therefore, we did not use perform IS on the MH control sample because the protein concentration in MH vitreous humor was roughly one tenth of than in PDR vitreous humor (MH protein concentration was 0.47 μg/μl, and PDR concentration was 4.13 μg/μl). Consequently, larger samples quantities should be obtained or a more sensitive instrument used to identify low abundance proteins in MH vitreous.

Of the 5 proteins that were identified in low abundance PDR gel, only two proteins (hemopexin and ARL6IP4) were detected in low abundance PDR gel (FIG. 2) and not in the other two gels (high abundance PDR gel and the non-IS-treated PDR gel). No new proteins were identified in low abundance PDR protein gel, but the 2-DE gel image of low abundance PDR proteins differed from that of non-IS-treated PDR proteins, which suggests the possibility that more low abundance proteins would have been be identified in the enriched fraction that had the detection limit of the method lower.

(2) Vitreous Protein Identification Using Nano LC-MALDI-MS/MS

In order to detect low abundance proteins in the PDR and control MH samples, we performed nano LC fractionation and protein identification using off-line nano LC-MALDI-MS/MS.

The 2-DE gel pattern of high abundance proteins in the IS-depleted PDR sample was similar to that in the corresponding non-IS-depleted PDR sample, which suggests that high abundance proteins account for most protein in vitreous humor. Therefore, we decided to use a relatively mild depletion method to prepare the depleted PDR vitreous sample, i.e., to deplete the PDR sample for nano LC-MALDI-MS/MS, we used a Calbiochem kit to remove only the two most abundant proteins, i.e., albumin and IgG.

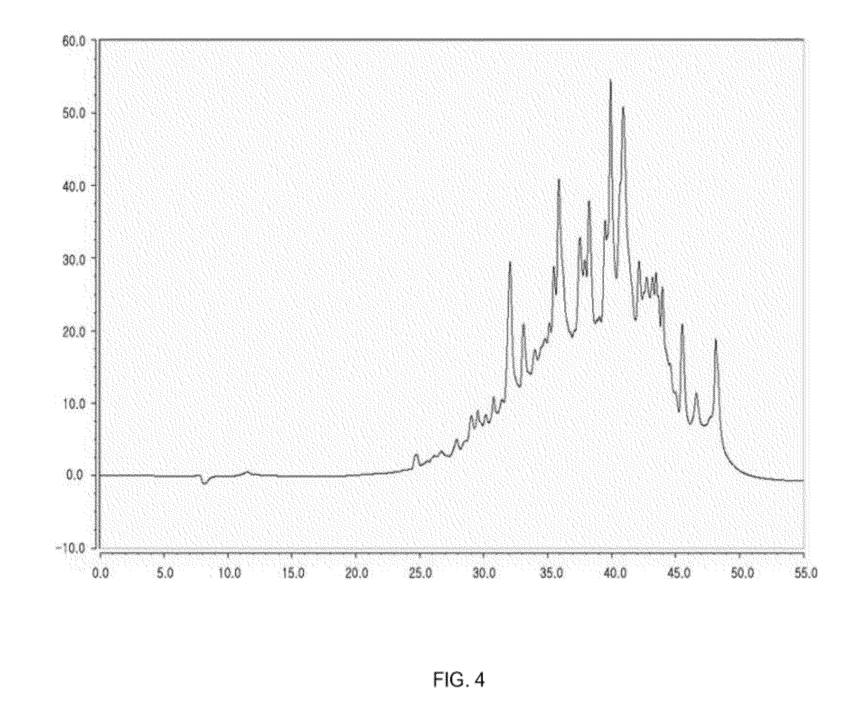

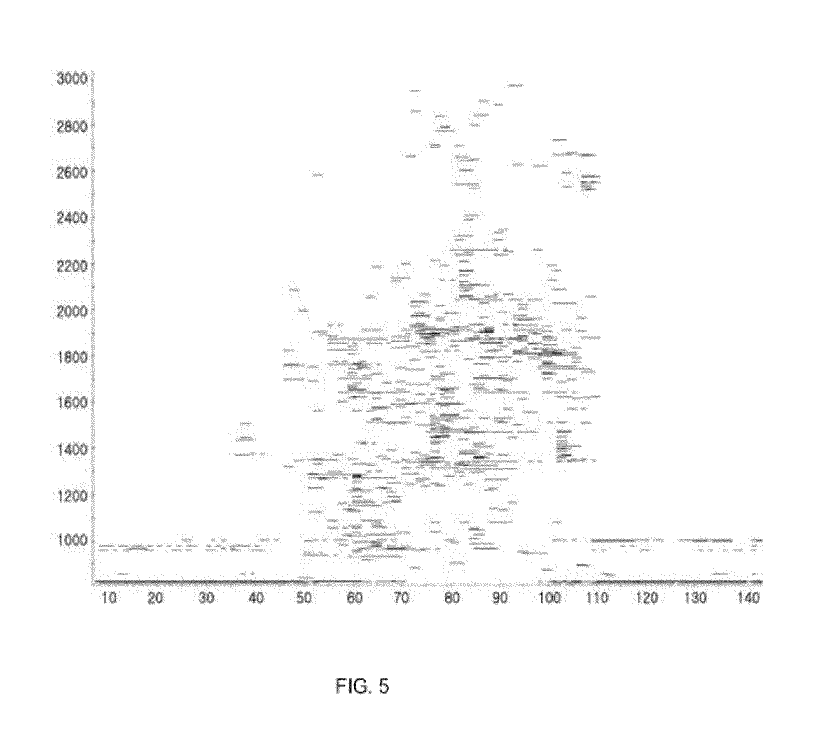

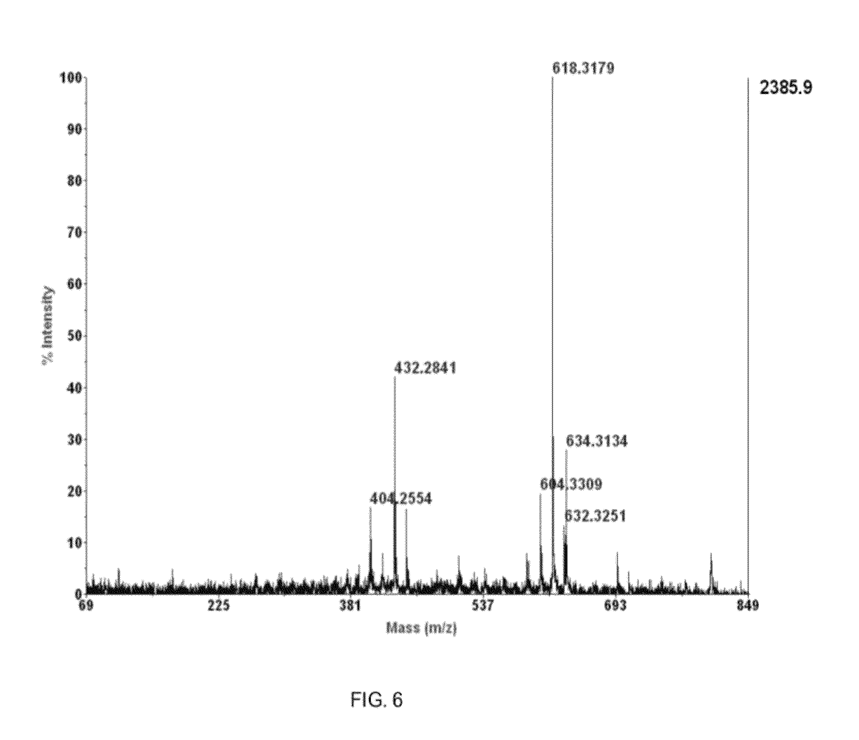

The prepared PDR, albumin/IgG depleted PDR, and control MH vitreous samples were run in SDS-PAGE gel, and gels were subsequently sliced evenly into 16 fractions (FIG. 3). After in-gel trypsin digestion, peptides in 20 μl of 0.1% TFA solution were injected into a nano LC equipped with autosampler using a 20 μl sample loop. The injected peptides were subject to nano LC separation 16 times and every nano LC run was followed by a blank run to avoid cross contamination. Peptides eluted from the nano LC were collected on a MALDI target plate (FIG. 4) and analyzed in MS/MS mode (FIGS. 5 and 6) and search results were revalidated using PeptideProphet and ProteinProphet in TPP.

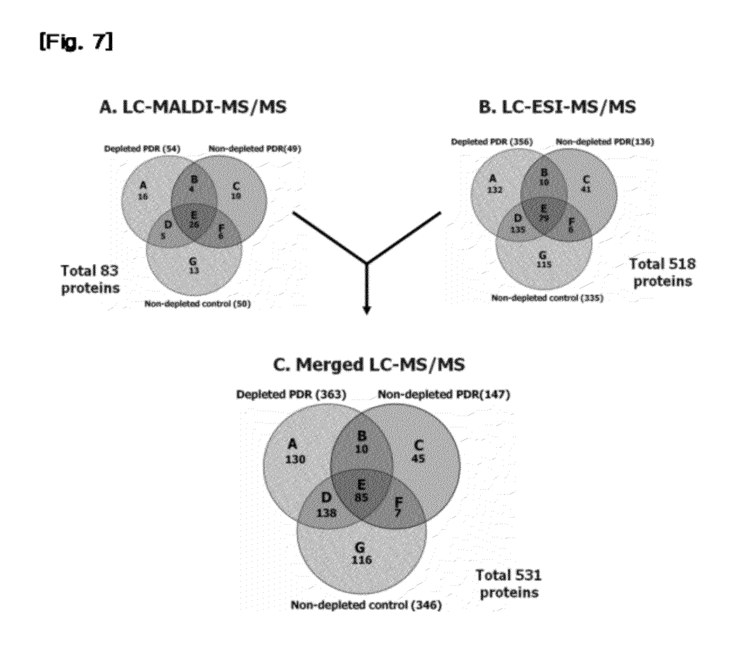

As a result (FIG. 7A), 54 proteins were identified in the albumin/IgG depleted PDR sample and 49 in the non-depleted PDR sample. In the control sample, 50 proteins were identified. In total, 83 proteins were identified in these three vitreous samples. A Venn diagram of the identified proteins is provided in FIG. 7A.

We carried out database searches using the NCBInr database (updated 20 Feb., 2007) and the IPI database (v3.24) for the 2-DE and LC-MALDI-MS/MS experiments. The result obtained from the NCBInr database are not included (data not shown), since it provoked data redundancy and complexity. Consequently, we used only the IPI database for reasons of experimental efficiency in this proteomics study.

(3) Vitreous Protein Identification Using Nano LC-ESI-MS/MS

To increase protein identification, we employed a complementary analytical platform, namely, nano LC-ESI-MS/MS. As a result of our nano LC-ESI-MS/MS experiment (FIG. 7B), 356 proteins were identified in albumin/IgG depleted PDR vitreous humor and 136 proteins in non-depleted PDR vitreous humor. 335 proteins were identified in the control MH vitreous sample. In total 518 proteins were identified in the non-depleted PDR, albumin/IgG depleted PDR, and control MH vitreous samples using LC-ESI-MS/MS (FIG. 7B). 183 (A, B, C of the Venn diagram) of the 518 proteins were found to be present only in PDR vitreous and 115 proteins (G of the Venn diagram) only in control vitreous. 220 proteins are present in the overlapping region of the Venn diagram (D, E, F of the Venn diagram).

(4) Identified Protein Lists from LC-MALDI-MS/MS and LC-ESI-MS/MS

The proteins identified using these two different ionization methods (MALDI and ESI) were combined to generate a collective vitreous proteome. 83 proteins identified by LC-MALDI-MS/MS and 518 proteins identified by LC-ESI-MS/MS generated a merged vitreous proteome profile consisting of 531 proteins (FIG. 7C). The identified protein lists from these two LC-MS/MS experiments included all proteins identified by 2-DE. The 531 proteins are as in the following Table 8 to 16.

TABLE 8

| | | | Newly | | | | | Probabil- | | |
|---|---|---|---|---|---|---|---|---|---|---|
| | | Sub- | detected | Detected | | | | ity | Number | Total |
| Venn | Total | group | in | in | | | | of | of | number |
| diagram | num- | num- | vitreous | plasma | | IPI accession | | Protein | unique | of |
| location | ber | ber | prote | proteome | Protein name | number | Method | Prophet | peptides | peptides |
| A | 1 | 1 | * | | 101 KDA PROTEIN | IPI00760855 | LTQ | 1 | 20 | 75 |
| | 2 | 2 | * | | 13 kDa protein | IPI00743473 | LTQ | 0.9 | 1 | 1 |
| | 3 | 3 | * | | 14-3-3 protein epsilon | IPI00000816 | LTQ | 1 | 4 | 4 |
| | 4 | 4 | * | * | 16 kDa protein | IPI00218733 | LTQ | 0.94 | 1 | 2 |
| | 5 | 5 | * | * | 184 KDA PROTEIN | IPI00303313 | LTQ | 1 | 4 | 4 |
| | 6 | 6 | * | | 57 kDa protein | IPI00383111 | LTQ | 1 | 13 | 41 |
| | 7 | 7 | * | | 97 KDA PROTEIN | IPI00794184 | LTQ | 1 | 76 | 366 |
| | 8 | 8 | * | * | Adiponectin precursor | IPI00020019 | LTQ | 1 | 2 | 4 |
| | 9 | 9 | * | | ADP-ribosylation factor 1 | IPI00215914 | LTQ | 1 | 2 | 2 |
| | 10 | 10 | * | | ALPHA3A | IPI00377045 | LTQ | 1 | 2 | 2 |
| | 11 | 11 | | | Amyloid lambda 6 light chain variable region | IPI00386839 | LTQ | 1 | 1 | 2 |
| | | | | | SAR (Fragment) | | | | | |
| | 12 | 12 | * | | ANNEXIN A2 ISOFORM 1 | IPI00418169 | LTQ | 0.9 | 2 | 2 |
| | 13 | 13 | | | ANTITHROMBIN III VARIANT | IPI00165421 | LTQ | 1 | 4 | 14 |
| | 14 | 14 | | * | Apolipoprotein C-III precursor | IPI00021857 | LTQ | 0.94 | 1 | 1 |
| | 15 | 15 | | * | apolipoprotein F precursor | IPI00299435 | LTQ | 0.94 | 1 | 1 |
| | 16 | 16 | | * | Apolipoprotein M | IPI00030739 | LTQ | 1 | 8 | 14 |
| | 17 | 17 | | | Beta crystallin A4 | IPI00419283 | LTQ | 1 | 7 | 8 |
| | 18 | 18 | * | | Beta-hexosaminidase beta chain precursor | IPI00012585 | LTQ | 1 | 2 | 2 |
| | 19 | 19 | * | | Biglycan precursor | IPI00010790 | LTQ | 1 | 2 | 2 |
| | 20 | 20 | | * | C4B-BINDING PROTEIN ALPHA CHAIN | IPI00021727 | LTQ | 0.94 | 1 | 1 |
| | | | | | PRECURSOR | | | | | |
| | 21 | 21 | * | | Calcium/calmodulin-dependent 3′,5′-cyclic | IPI00005592 | LTQ | 1 | 2 | 3 |
| | | | | | nucleotide phosphodiesterase 1B | | | | | |
| | 22 | 22 | * | * | CALMODULIN-LIKE PROTEIN 5 | IPI00021536 | LTQ | 0.94 | 1 | 2 |
| | 23 | 23 | | | Catalase | IPI00465436 | LTQ | 0.92 | 1 | 1 |
| | 24 | 24 | * | | CD59 glycoprotein precursor | IPI00011302 | LTQ | 1 | 4 | 8 |
| | 25 | 25 | * | | CDNA FLJ25678 fis, clone TST04067, highly | IPI00017672 | LTQ | 1 | 4 | 5 |
| | | | | | similar to PURINE NUCLEOSIDE | | | | | |
| | | | | | PHOSPHORYLASE | | | | | |
| | 26 | 26 | * | | CDNA FLJ41981 fis, clone SMINT2011888, | IPI00784830 | LTQ | 1 | 2 | 3 |
| | | | | | highly similar to Protein Tro alpha1 H, myeloma | | | | | |
| | 27 | 27 | * | * | Cholinesterase precursor | IPI00025864 | LTQ | 1 | 2 | 2 |
| | 28 | 28 | | * | Coagulation factor IX precursor | IPI00296176 | LTQ | 0.94 | 1 | 3 |
| | 29 | 29 | * | | Cofilin-1 | IPI00012011 | LTQ | 1 | 2 | 2 |
| | 30 | 30 | | * | Collagen alpha-1(VI) chain precursor | IPI00291136 | LTQ | 1 | 3 | 4 |
| | 31 | 31 | | | Collagen alpha-2(I) chain precursor | IPI00304962 | LTQ | 1 | 3 | 3 |
| | 32 | 32 | | * | Complement C1q subcomponent subunit A | IPI00022392 | LTQ | 1 | 2 | 2 |
| | | | | | precursor | | | | | |
| | 33 | 33 | | | Complement C3 precursor (Fragment) | IPI00783987 | LTQ | 1 | 103 | 640 |
| | 34 | 34 | | * | Complement C4-A precursor | IPI00032258 | LTQ | 1 | 11 | 74 |
| | 35 | 35 | * | | Corneodesmosin precursor | IPI00386809 | LTQ | 0.94 | 1 | 1 |
| | 36 | 36 | * | | Dermatopontin precursor | IPI00292130 | LTQ | 1 | 3 | 3 |
| | 37 | 37 | | * | Dystroglycan precursor | IPI00028911 | LTQ | 1 | 3 | 3 |
| | 38 | 38 | * | | E3 UBIQUITIN-PROTEIN LIGASE HECTD1 | IPI00328911 | LTQ | 0.92 | 2 | 5 |
| | 39 | 39 | * | | Endothelial protein C receptor precursor | IPI00009276 | LTQ | 1 | 2 | 3 |
| | 40 | 40 | * | | FERRITIN HEAVY CHAIN | IPI00554521 | LTQ | 0.99 | 1 | 1 |
| | 41 | 41 | * | | FERRITIN LIGHT POLYPEPTIDE VARIANT | IPI00796538 | LTQ | 1 | 10 | 19 |
| | 42 | 42 | * | * | Fetuin-B precursor | IPI00005439 | LTQ | 1 | 5 | 5 |
| | 43 | 43 | * | | FIBRONECTIN 1 ISOFORM 4 | IPI00414283 | LTQ | 0.98 | 1 | 1 |
| | | | | | PREPROPROTEIN | | | | | |
| | 44 | 44 | * | | Fructose-bisphosphate aldolase C | IPI00418262 | LTQ | 0.98 | 2 | 2 |
| | 45 | 45 | | | Gamma crystallin C | IPI00220282 | LTQ | 1 | 5 | 5 |
| | 46 | 46 | | | Gamma crystallin D | IPI00215881 | LTQ | 1 | 2 | 3 |
| | 47 | 47 | * | * | Gamma-glutamyl hydrolase precursor | IPI00023728 | LTQ | 1 | 5 | 6 |
| | 48 | 48 | * | | Gastrokine-1 precursor | IPI00021342 | LTQ | 1 | 4 | 5 |
| | 49 | 49 | | * | Glutathione S-transferase P | IPI00219757 | LTQ | 0.94 | 1 | 1 |
| | 50 | 50 | | * | Glyceraldehyde-3-phosphate dehydrogenase | IPI00219018 | LTQ | 1 | 5 | 12 |
| | 51 | 51 | * | * | Growth/differentiation factor 8 precursor | IPI00023751 | LTQ | 0.94 | 1 | 1 |
| | 52 | 52 | | | Hemoglobin subunit gamma-1 | IPI00220706 | LTQ | 1 | 4 | 5 |
| | 53 | 53 | * | * | Hepatocyte growth factor activator precursor | IPI00029193 | LTQ | 1 | 2 | 2 |
| | 54 | 54 | * | | Hornerin | IPI00398625 | LTQ | 1 | 1 | 2 |
| | 55 | 55 | | | HYPOTHETICAL PROTEIN | IPI00784519 | LTQ | 1 | 1 | 1 |
| | 56 | 56 | | | | IPI00784894 | LTQ | 1 | 4 | 6 |
| | 57 | 57 | | | Hypothetical protein DKFZp686C02220 | IPI00423461 | LTQ | 1 | 2 | 6 |
| | | | | | (Fragment) | | | | | |
| | 58 | 58 | | | Hypothetical protein DKFZp686K04218 | IPI00384952 | LTQ | 0.93 | 1 | 3 |
| | | | | | (Fragment) | | | | | |
| | 59 | 59 | | | HYPOTHETICAL PROTEIN | IPI00423462 | LTQ | 0.99 | 1 | 1 |
| | | | | | DKFZP686K18196 (FRAGMENT) | | | | | |
| | 60 | 60 | | * | hypothetical protein LOC80208 | IPI00101923 | LTQ | 1 | 3 | 4 |
| | 61 | 61 | * | | Hypoxanthine-guanine phosphoribosyltransferase | IPI00218493 | LTQ | 0.93 | 1 | 1 |
| | 62 | 62 | | * | Ig kappa chain V-III region VH precursor | IPI00024138 | LTQ | 0.9 | 1 | 1 |

TABLE 9

| | | | Newly | | | | | Proba- | | |
|---|---|---|---|---|---|---|---|---|---|---|
| | | Sub- | detected | Detected | | | | bility | Number | Total |
| Venn | Total | group | in | in | | | | of | of | number |
| diagram | num- | num- | vitreous | plasma | | IPI accession | | Protein | unique | of |
| location | ber | ber | proteome | proteome | Protein name | number | Method | Prophet | peptides | peptides |
| | 63 | 63 | | | Ig lambda chain V-III region SH | IPI00382436 | LTQ | 0.94 | 1 | 2 |
| | 64 | 64 | | | IGHM PROTEIN | IPI00549291 | LTQ | 1 | 15 | 25 |
| | 65 | 65 | | | IGKC PROTEIN | IPI00761125 | LTQ | 1 | 1 | 1 |
| | 66 | 66 | | | IGLV6-57 protein | IPI00419442 | LTQ | 1 | 1 | 1 |
| | 67 | 67 | | * | immunoglobulin J chain | IPI00178926 | LTQ | 1 | 2 | 2 |
| | 68 | 68 | | | Immunoglobulin lambda-like | IPI00013438 | LTQ | 0.94 | 1 | 1 |
| | | | | | polypeptide 1 precursor | | | | | |
| | 69 | 69 | | * | Insulin-like growth factor-binding | IPI00297284 | LTQ | 0.96 | 1 | 1 |
| | | | | | protein 2 precursor | | | | | |
| | 70 | 70 | | | Insulin-like growth factor-binding | IPI00029236 | LTQ | 1 | 2 | 3 |
| | | | | | protein 5 precursor | | | | | |
| | 71 | 71 | | * | Inter-alpha-trypsin inhibitor heavy | IPI00028413 | LTQ | 1 | 7 | 9 |
| | | | | | chain H3 precursor | | | | | |
| | 72 | 72 | * | * | Intercellular adhesion molecule 2 | IPI00009477 | LTQ | 0.93 | 1 | 1 |
| | | | | | precursor | | | | | |
| | 73 | 73 | * | | Isoform 1 of Arginase-1 | IPI00291560 | LTQ | 1 | 2 | 2 |
| | 74 | 74 | | * | Isoform 1 of Collagen alpha-1(III) chain | IPI00021033 | LTQ | 0.92 | 1 | 1 |
| | | | | | precursor | | | | | |
| | 75 | 75 | | | Isoform 1 of Complement C1q tumor | IPI00008860 | LTQ | 0.94 | 1 | 1 |
| | | | | | necrosis factor-related protein 3 | | | | | |
| | | | | | precursor | | | | | |
| | 76 | 76 | * | * | Isoform 1 of Contactin-4 precursor | IPI00178854 | LTQ | 0.93 | 1 | 1 |
| | 77 | 77 | * | * | Isoform 1 of C-reactive protein | IPI00022389 | LTQ | 1 | 2 | 2 |
| | | | | | precursor | | | | | |
| | 78 | 78 | * | * | Isoform 1 of Ficolin-3 precursor | IPI00293925 | LTQ | 1 | 7 | 7 |
| | 79 | 79 | | | Isoform 1 of Haptoglobin-related | IPI00477597 | LTQ | 1 | 13 | 20 |
| | | | | | protein precursor | | | | | |
| | 80 | 80 | * | * | Isoform 1 of Mannan-binding lectin | IPI00294713 | LTQ | 1 | 3 | 5 |
| | | | | | serine protease 2 precursor | | | | | |
| | 81 | 81 | * | * | Isoform 1 of Multiple epidermal growth | IPI00027310 | LTQ | 1 | 2 | 2 |
| | | | | | factor-like domains 8 | | | | | |
| | 82 | 82 | * | | Isoform 1 of Phosphatidylinositol-glycan- | IPI00299503 | LTQ | 1 | 5 | 5 |
| | | | | | specific phospholipase D precursor | | | | | |
| | 83 | 83 | * | | ISOFORM 1 OF PHOSPHOLIPID | IPI00643034 | LTQ | 1 | 4 | 5 |
| | | | | | TRANSFER PROTEIN PRECURSOR | | | | | |
| | 84 | 84 | * | * | Isoform 1 of Plexin domain-containing | IPI00044369 | LTQ | 0.94 | 1 | 1 |
| | | | | | protein 2 precursor | | | | | |
| | 85 | 85 | * | * | Isoform 1 of Probable helicase senataxin | IPI00142538 | LTQ | 0.99 | 2 | 2 |
| | 86 | 86 | | * | Isoform 1 of Scavenger receptor cysteine- | IPI00104074 | LTQ | 1 | 3 | 4 |
| | | | | | rich type 1 protein M130 precursor | | | | | |
| | 87 | 87 | * | * | Isoform A of Proteoglycan-4 precursor | IPI00024825 | LTQ | 1 | 2 | 2 |
| | 88 | 88 | | * | Isoform Long of Complement factor H- | IPI00006154 | LTQ | 0.91 | 1 | 1 |
| | | | | | related protein 2 precursor | | | | | |
| | 89 | 89 | * | * | Kallistatin precursor | IPI00328609 | LTQ | 1 | 3 | 3 |
| | 90 | 90 | | | KERATIN, TYPE I CYTOSKELETAL 17 | gi\|547751\|sp\|Q [symbol] | LTQ | 1 | 5 | 6 |
| | | | | | (CYTOKERATIN 17) (K17) (CK 17) (39 | | | | | |
| | 91 | 91 | | | Keratin-80 | IPI00375843 | LTQ | 1 | 3 | 3 |
| | 92 | 92 | * | * | Lipopolysaccharide-binding protein | IPI00032311 | LTQ | 1 | 5 | 5 |
| | | | | | precursor | | | | | |
| | 93 | 93 | * | | Lithostathine 1 alpha precursor | IPI00009027 | LTQ | 1 | 4 | 4 |
| | 94 | 94 | * | * | Macrophage colony-stimulating factor 1 | IPI00011218 | LTQ | 0.94 | 1 | 1 |
| | | | | | receptor precursor | | | | | |
| | 95 | 95 | * | * | MAN1A1 PROTEIN | IPI00291641 | LTQ | 0.93 | 1 | 1 |
| | 96 | 96 | | | Microfibril-associated glycoprotein 4 | IPI00022792 | LTQ | 1 | 3 | 3 |
| | | | | | precursor | | | | | |
| | 97 | 97 | * | * | MIMECAN PRECURSOR | IPI00025465 | LTQ | 0.94 | 1 | 1 |
| | 98 | 98 | * | | MUCIN-5B PRECURSOR | IPI00384897 | LTQ | 1 | 5 | 6 |
| | 99 | 99 | * | * | Multimerin-2 precursor | IPI00015525 | LTQ | 1 | 4 | 7 |
| | 100 | 100 | * | * | Myocilin precursor | IPI00019190 | LTQ | 1 | 5 | 5 |
| | 101 | 101 | * | | Myoglobin | IPI00217493 | LTQ | 0.93 | 1 | 1 |
| | 102 | 102 | * | | Neurexin 3-alpha | IPI00216728 | LTQ | 1 | 3 | 3 |
| | 103 | 103 | | * | Neutrophil defensin 1 precursor | IPI00005721 | LTQ | 0.93 | 1 | 1 |
| | 104 | 104 | | * | Neutrophil gelatinase-associated lipocalin | IPI00299547 | LTQ | 1 | 4 | 6 |
| | | | | | precursor | | | | | |
| | 105 | 105 | * | * | Nidogen-2 precursor | IPI00028908 | LTQ | 0.94 | 1 | 1 |
| | 106 | 106 | | | BETA CASEIN PRECURSOR. - | CASB_BOVIN | LTQ | 0.96 | 1 | 1 |
| | | | | | BOS TAURUS (BOVINE) | | | | | |
| | 107 | 107 | | | VATVSLPR-like Promega trypsin | Trypa1\|PromTArt1 | LTQ | 1 | 2 | 45 |
| | | | | | artifact 1 (871.1) xATVSLPR | | | | | |
| | 108 | 108 | * | * | PEPTIDYL-PROLYL CIS-TRANS | IPI00024129 | LTQ | 0.9 | 1 | 1 |
| | | | | | ISOMERASE C | | | | | |
| | 109 | 109 | | * | Peroxiredoxin-2 | IPI00027350 | LTQ | 1 | 6 | 8 |
| | 110 | 110 | * | | Phosphatidylethanolamine-binding | IPI00219446 | LTQ | 1 | 4 | 5 |
| | | | | | protein 1 | | | | | |
| | 111 | 111 | | * | Phosphoglycerate kinase 1 | IPI00169383 | LTQ | 1 | 2 | 2 |
| | 112 | 112 | * | * | Pregnancy zone protein precursor | IPI00025426 | LTQ | 1 | 20 | 149 |
| | 113 | 113 | | * | protease inhibitor 16 precursor | IPI00301143 | LTQ | 1 | 2 | 2 |
| | 114 | 114 | * | | Protein DJ-1 | IPI00298547 | LTQ | 0.94 | 1 | 1 |
| | 115 | 115 | * | | Pseudogene candidate | IPI00454869 | LTQ | 1 | 2 | 3 |
| | 116 | 116 | * | | Rho GDP-dissociation inhibitor 2 | IPI00003817 | LTQ | 1 | 2 | 2 |
| | 117 | 117 | * | * | Serpin B4 | IPI00010303 | LTQ | 0.93 | 1 | 1 |
| | 118 | 118 | | | SERUM ALBUMIN PRECURSOR | gi\|113574\|sp\|P [symbol] | LTQ | 1 | 9 | 20 |
| | 119 | 119 | | | similar to C3 and PZP-like, alpha-2- | IPI00249915 | LTQ | 1 | 2 | 2 |
| | | | | | macroglobulin domain containing 8 | | | | | |
| | 120 | 120 | | | Similar to Ig kappa chain V-IV region | IPI00026197 | LTQ | 0.94 | 4 | 9 |
| | | | | | STH | | | | | |
| | 121 | 121 | | | SINGLE-CHAIN FV (FRAGMENT) | IPI00748998 | LTQ | 1 | 3 | 3 |
| | 122 | 122 | * | * | SUPEROXIDE DISMUTASE [MN], | IPI00022314 | LTQ | 0.94 | 1 | 1 |
| | | | | | MITOCHONDRIAL PRECURSOR | | | | | |
| | 123 | 123 | * | * | Thioredoxin | IPI00216298 | LTQ | 0.93 | 1 | 1 |

[symbol]

indicates data missing or illegible when filed

TABLE 10

| | | | Newly | | | | | Proba- | | |
|---|---|---|---|---|---|---|---|---|---|---|
| | | Sub- | detected | Detected | | | | bility | Number | Total |
| Venn | Total | group | in | in | | | | of | of | number |
| diagram | num- | num- | vitreous | plasma | | IPI accession | | Protein | unique | of |
| location | ber | ber | proteome | proteome | Protein name | number | Method | Prophet | peptides | peptides |
| | 124 | 124 | * | * | Thyroxine-binding globulin precursor | IPI00292946 | LTQ | 1 | 21 | 56 |
| | 125 | 125 | * | | TRIOSEPHOSPHATE ISOMERASE 1 | IPI00465028 | LTQ | 1 | 6 | 7 |
| | | | | | VARIANT | | | | | |
| | 126 | 126 | * | * | UNCHARACTERIZED PROTEIN C7ORF24 | IPI00031564 | LTQ | 0.94 | 1 | 1 |
| | 127 | 127 | * | | V1-17 protein | IPI00045547 | LTQ | 1 | 3 | 6 |
| | 128 | 128 | * | | V1-5 protein (Fragment) | IPI00553215 | LTQ | 0.94 | 1 | 1 |
| | 129 | 129 | * | * | von Willebrand factor precursor | IPI00023014 | LTQ | 1 | 3 | 3 |
| | 130 | 130 | * | | WSB-1 ISOFORM | IPI00383777 | LTQ | 0.91 | 1 | 1 |
| B | 131 | 1 | | | Alpha crystallin B chain | IPI00021369 | LTQ | 1 | 20 | 104 |
| | 132 | 2 | | * | Apolipoprotein B-100 precursor | IPI00022229 | LTQ | 1 | 36 | 43 |
| | 133 | 3 | | | Collagen alpha-1(I) chain precursor | IPI00297646 | LTQ | 1 | 4 | 4 |
| | 134 | 4 | | * | Fibrinogen beta chain precursor | IPI00298497 | MAL | 1 | 37 | 120 |
| | 135 | 5 | | | Haptoglobin precursor | IPI00641737 | LTQ | 1 | 18 | 363 |
| | 136 | 6 | | | Ig kappa chain V-I region Mev | IPI00387105 | LTQ | 0.93 | 1 | 2 |
| | 137 | 7 | | | Ig kappa chain V-II region TEW | IPI00736885 | LTQ | 1 | 4 | 8 |
| | 138 | 8 | | | IGLV3-25 PROTEIN | IPI00550162 | LTQ | 1 | 3 | 193 |
| | 139 | 9 | | * | Isoform 1 of Fibronectin precursor | IPI00022418 | MAL | 0.99 | 1 | 1 |
| | 140 | 10 | | * | Serum amyloid P-component precursor | IPI00022391 | MAL | 1 | 5 | 9 |
| C | 141 | 1 | | | (S43646) cytokeratin 2, CK 2 [human, | gi\|254622\|bbs\| | LTQ | 1 | 9 | 38 |
| | | | | | epidermis, Peptide, 645 aa] [Homo sapiens] | | | | | |
| | 142 | 2 | * | | 10 kDa protein | IPI00740756 | LTQ | 1 | 2 | 17 |
| | 143 | 3 | * | | 25 kDa protein | IPI00448800 | LTQ | 1 | 2 | 125 |
| | 144 | 4 | * | * | 272 KDA PROTEIN | IPI00219299 | LTQ | 0.91 | 2 | 3 |
| | 145 | 5 | * | | 330 kDa protein | IPI00163866 | LTQ | 0.99 | 2 | 2 |
| | 146 | 6 | * | | 3′-5′ exoribonuclease CSL4 homolog | IPI00032823 | LTQ | 0.95 | 1 | 1 |
| | 147 | 7 | * | | ACF7 PROTEIN | IPI00183169 | LTQ | 0.91 | 2 | 2 |
| | 148 | 8 | * | | Actin, aortic smooth muscle | IPI00008603 | LTQ | 0.99 | 1 | 1 |
| | 149 | 9 | | | ALPHA-A-CRYSTALLIN | IPI00795775 | LTQ | 0.98 | 1 | 1 |
| | 150 | 10 | * | * | ATP-binding cassette, sub-family A, member 2 | IPI00307592 | LTQ | 0.97 | 2 | 8 |
| | | | | | isoform a | | | | | |
| | 151 | 11 | * | | BONE MORPHOGENETIC PROTEIN | IPI00005731 | LTQ | 0.93 | 2 | 2 |
| | | | | | RECEPTOR TYPE IA PRECURSOR | | | | | |
| | 152 | 12 | | * | Brain-specific serine protease 4 precursor | IPI00005467 | LTQ | 0.99 | 2 | 2 |
| | 153 | 13 | | * | CADHERIN-20 PRECURSOR | IPI00307612 | LTQ | 1 | 2 | 3 |
| | 154 | 14 | * | | CDNA: FLJ21459 fis, clone COL04714 | IPI00001606 | LTQ | 0.99 | 2 | 29 |
| | 155 | 15 | * | * | CENTROMERE PROTEIN F | IPI00027157 | LTQ | 0.97 | 2 | 2 |
| | 156 | 16 | * | | CRYPTOCHROME-1 | IPI00002540 | LTQ | 0.91 | 2 | 4 |
| | 157 | 17 | * | * | Dpy-19-like protein 1 | IPI00007461 | LTQ | 1 | 2 | 31 |
| | 158 | 18 | * | * | EXOCYST COMPLEX COMPONENT 8 | IPI00028264 | LTQ | 0.98 | 2 | 2 |
| | 159 | 19 | | | Hypothetical protein DKFZp686E23209 | IPI00784942 | LTQ | 1 | 7 | 222 |
| | 160 | 20 | | | Hypothetical protein DKFZp686I04196 | IPI00399007 | MAL | 1 | 4 | 17 |
| | | | | | (Fragment) | | | | | |
| | 161 | 21 | | | Ig kappa chain V-I region OU | IPI00387098 | LTQ | 1 | 2 | 4 |
| | 162 | 22 | | | IG KAPPA CHAIN V-IV REGION B17 | IPI00386133 | LTQ | 1 | 6 | 102 |
| | | | | | PRECURSOR | | | | | |
| | 163 | 23 | | * | IGHA1 PROTEIN | IPI00061977 | MAL | 1 | 3 | 5 |
| | 164 | 24 | | | | IPI00744561 | LTQ | 1 | 7 | 28 |
| | 165 | 25 | | | IGL@ PROTEIN | IPI00658130 | MAL | 1 | 5 | 15 |
| | 166 | 26 | * | | ISOFORM 1 OF ALANINE | IPI00152432 | LTQ | 0.94 | 2 | 2 |
| | | | | | AMINOTRANSFERASE 2 | | | | | |
| | 167 | 27 | * | * | ISOFORM 1 OF GRIP AND COILED-COIL | IPI00005631 | LTQ | 0.92 | 2 | 2 |
| | | | | | DOMAIN-CONTAINING PROTEIN 2 | | | | | |
| | 168 | 28 | * | | ISOFORM 1 OF PROBABLE E3 | IPI00333067 | LTQ | 0.97 | 2 | 2 |
| | | | | | UBIQUITIN-PROTEIN LIGASE HERC4 | | | | | |
| | 169 | 29 | * | | ISOFORM 1 OF | IPI00069084 | LTQ | 0.9 | 2 | 3 |
| | | | | | TRANSFORMATION/TRANSCRIPTION | | | | | |
| | | | | | DOMAIN-ASSOCIATED PROTEIN | | | | | |
| | 170 | 30 | * | | Isoform 1 of Uncharacterized protein C9orf84 | IPI00658203 | LTQ | 0.99 | 2 | 4 |
| | 171 | 31 | * | * | ISOFORM 2 OF CROSSOVER JUNCTION | IPI00073193 | LTQ | 0.94 | 2 | 4 |
| | | | | | ENDONUCLEASE EME1 | | | | | |
| | 172 | 32 | * | * | ISOFORM 4 OF NESPRIN-1 | IPI00247295 | LTQ | 0.9 | 3 | 3 |
| | 173 | 33 | * | * | Junctional adhesion molecule A precursor | IPI00001754 | LTQ | 0.99 | 2 | 3 |
| | 174 | 34 | | | KERATIN, TYPE I CYTOSKELETAL 10 | gi\|547749\|sp\|P | LTQ | 1 | 15 | 242 |
| | | | | | (CYTOKERATIN 10) (K10) (CK 10) | | | | | |
| | 175 | 35 | | | KERATIN, TYPE II MICROFIBRILLAR, | gi\|125116\|sp\|P | LTQ | 1 | 3 | 16 |
| | | | | | COMPONENT 7C | | | | | |
| | 176 | 36 | * | * | Mucin 5 (Fragment) | IPI00103397 | LTQ | 0.97 | 2 | 3 |
| | 177 | 37 | | | Myosin-reactive immunoglobulin kappa chain | IPI00384401 | MAL | 0.98 | 2 | 4 |
| | | | | | variable region (Fragment) | | | | | |
| | 178 | 38 | * | * | POTASSIUM/SODIUM | IPI00031506 | LTQ | 0.98 | 2 | 3 |
| | | | | | HYPERPOLARIZATION-ACTIVATED | | | | | |
| | | | | | CYCLIC NUCLEOTIDE-GATED | | | | | |
| | | | | | CHANNEL 1 | | | | | |
| | 179 | 39 | | | ProSAAS precursor | IPI00002280 | LTQ | 0.97 | 2 | 4 |
| | 180 | 40 | * | * | PROTEIN BASSOON | IPI00020153 | LTQ | 0.92 | 2 | 2 |

TABLE 11

| | | | Newly | | | | | Proba- | | |
|---|---|---|---|---|---|---|---|---|---|---|
| | | Sub- | detected | Detected | | | | bility | Number | Total |
| Venn | Total | group | in | in | | | | of | of | number |
| diagram | num- | num- | vitreous | plasma | | IPI accession | | Protein | unique | of |
| location | ber | ber | proteome | proteome | Protein name | number | Method | Prophet | peptides | peptides |
| | 181 | 41 | * | | SIMILAR TO GENERAL TRANSCRIPTION | IPI00736974 | LTQ | 0.97 | 2 | 2 |
| | | | | | FACTOR II-I REPEAT DOMAIN- | | | | | |
| | | | | | CONTAINING PROTEIN 1 (GTF2I REPEAT | | | | | |
| | | | | | DOMAIN-CONTAINING PROTEIN 1) | | | | | |
| | | | | | (MUSCLE TFII-I REPEAT DOMAIN- | | | | | |
| | | | | | CONTAINING PROTEIN 1) (GENERAL | | | | | |
| | | | | | TRANSCRIPTION FACTOR III) (SLOW- | | | | | |
| | | | | | MUSCLE-FIBER ENHANCER BINDING PRO | | | | | |
| | 182 | 42 | * | | Structural maintenance of chromosomes protein | IPI00479260 | LTQ | 1 | 3 | 3 |
| | | | | | 1B | | | | | |
| | 183 | 43 | * | | Thyroid hormone receptor-associated protein 2 | IPI00400834 | LTQ | 0.98 | 2 | 4 |
| | 184 | 44 | * | | UNCHARACTERIZED PROTEIN C22ORF30 | IPI00643747 | LTQ | 1 | 2 | 5 |
| | 185 | 45 | * | * | Utrophin | IPI00009329 | LTQ | 0.99 | 3 | 3 |
| D | 186 | 1 | * | | 12 kDa protein | IPI00790473 | LTQ | 0.99 | 1 | 1 |
| | 187 | 2 | * | | 13 kDa protein | IPI00796830 | LTQ | 0.99 | 1 | 2 |
| | 188 | 3 | * | | 14-3-3 protein zeta/delta | IPI00021263 | LTQ | 0.96 | 1 | 1 |
| | 189 | 4 | * | | 26 kDa protein | IPI00738024 | LTQ | 1 | 4 | 9 |
| | 190 | 5 | * | | 61 kDa protein | IPI00373937 | LTQ | 0.96 | 1 | 1 |
| | 191 | 6 | * | | Acid ceramidase precursor | IPI00013698 | LTQ | 1 | 11 | 15 |
| | 192 | 7 | * | * | Actin, cytoplasmic 1 | IPI00021439 | LTQ | 1 | 9 | 12 |
| | 193 | 8 | * | | ADAMTS-1 precursor | IPI00005908 | LTQ | 1 | 3 | 3 |
| | 194 | 9 | | * | Afamin precursor | IPI00019943 | LTQ | 1 | 25 | 84 |
| | 195 | 10 | * | * | Alpha-2-antiplasmin precursor | IPI00029863 | LTQ | 1 | 18 | 62 |
| | 196 | 11 | | * | Amyloid-like protein 1 precursor | IPI00020012 | LTQ | 1 | 8 | 11 |
| | 197 | 12 | | * | Angiotensinogen precursor | IPI00032220 | MAL | 1 | 35 | 139 |
| | 198 | 13 | * | * | Basement membrane-specific heparan sulfate | IPI00024284 | LTQ | 1 | 28 | 32 |
| | | | | | proteoglycan core protein precursor | | | | | |
| | 199 | 14 | | | Beta crystallin B1 | IPI00216092 | MAL | 1 | 12 | 24 |
| | 200 | 15 | | | Beta crystallin B2 | IPI00218748 | MAL | 1 | 14 | 48 |
| | 201 | 16 | | | Beta crystallin S | IPI00554640 | MAL | 1 | 18 | 37 |
| | 202 | 17 | * | * | biotinidase precursor | IPI00218413 | LTQ | 1 | 9 | 24 |
| | 203 | 18 | | * | Carbonic anhydrase 2 | IPI00218414 | LTQ | 1 | 7 | 8 |
| | 204 | 19 | | | Carboxypeptidase E precursor | IPI00031121 | MAL | 1 | 12 | 20 |
| | 205 | 20 | | | Carboxypeptidase N subunit 2 precursor | IPI00479116 | LTQ | 1 | 5 | 5 |
| | 206 | 21 | | * | Cathepsin D precursor | IPI00011229 | MAL | 1 | 15 | 54 |
| | 207 | 22 | | | Cathepsin L precursor | IPI00012887 | LTQ | 1 | 5 | 5 |
| | 208 | 23 | | * | Cathepsin Z precursor | IPI00002745 | LTQ | 1 | 4 | 4 |
| | 209 | 24 | * | | CDNA FLJ14473 fis, clone MAMMA1001080, | IPI00386879 | LTQ | 1 | 3 | 3 |
| | | | | | highly similar to Homo sapiens SNC73 protein | | | | | |
| | | | | | (SNC73) mRNA | | | | | |
| | 210 | 25 | | * | Coagulation factor XII precursor | IPI00019581 | LTQ | 1 | 7 | 8 |
| | 211 | 26 | | * | Collagen alpha-2(IX) chain precursor | IPI00019088 | LTQ | 1 | 3 | 4 |
| | 212 | 27 | | * | Complement C1q subcomponent subunit C | IPI00022394 | LTQ | 1 | 3 | 4 |
| | | | | | precursor | | | | | |
| | 213 | 28 | | * | Complement C1r subcomponent precursor | IPI00296165 | LTQ | 1 | 7 | 7 |
| | 214 | 29 | | * | Complement C1s subcomponent precursor | IPI00017696 | LTQ | 1 | 9 | 9 |
| | 215 | 30 | | | complement component 1, q subcomponent, B | IPI00477992 | LTQ | 1 | 8 | 11 |
| | | | | | chain precursor | | | | | |
| | 216 | 31 | | * | Complement component C7 precursor | IPI00296608 | LTQ | 1 | 9 | 12 |
| | 217 | 32 | | * | Complement factor D precursor | IPI00019579 | LTQ | 1 | 3 | 3 |
| | 218 | 33 | * | * | Corticosteroid-binding globulin precursor | IPI00027482 | MAL | 1 | 14 | 28 |
| | 219 | 34 | * | * | Dermcidin precursor | IPI00027547 | LTQ | 1 | 3 | 11 |
| | 220 | 35 | * | * | desmocollin 1 isoform Dsc1b preproprotein | IPI00007425 | LTQ | 1 | 3 | 4 |
| | 221 | 36 | * | * | Desmoglein-1 precursor | IPI00025753 | LTQ | 1 | 6 | 7 |
| | 222 | 37 | | | Dickkopf-related protein 3 precursor | IPI00002714 | MAL | 1 | 11 | 40 |
| | 223 | 38 | * | | Dipeptidyl-peptidase 2 precursor | IPI00296141 | LTQ | 1 | 3 | 3 |
| | 224 | 39 | * | * | Endothelial cell-selective adhesion molecule | IPI00303161 | LTQ | 0.94 | 1 | 2 |
| | | | | | precursor | | | | | |
| | 225 | 40 | * | | Epididymal secretory protein E1 precursor | IPI00301579 | LTQ | 1 | 4 | 14 |
| | 226 | 41 | * | * | Extracellular superoxide dismutase [Cu—Zn] | IPI00027827 | LTQ | 1 | 5 | 6 |
| | | | | | precursor | | | | | |
| | 227 | 42 | * | * | Follistatin-related protein 5 precursor | IPI00008087 | LTQ | 1 | 16 | 20 |
| | 228 | 43 | | * | Galectin-3-binding protein precursor | IPI00023673 | LTQ | 1 | 6 | 9 |
| | 229 | 44 | | | Ganglioside GM2 activator precursor | IPI00018236 | LTQ | 0.96 | 1 | 1 |
| | 230 | 45 | * | * | Heparin cofactor 2 precursor | IPI00292950 | LTQ | 1 | 19 | 47 |
| | 231 | 46 | | | HYPOTHETICAL PROTEIN | IPI00550731 | LTQ | 1 | 2 | 3 |
| | 232 | 47 | | | | IPI00784865 | LTQ | 1 | 1 | 1 |
| | 233 | 48 | | | | IPI00784969 | LTQ | 1 | 2 | 6 |
| | 234 | 49 | | | HYPOTHETICAL PROTEIN | IPI00792115 | LTQ | 1 | 1 | 2 |
| | | | | | DKFZP686H17246 | | | | | |
| | 235 | 50 | | | Hypothetical protein LOC196463 | IPI00169285 | LTQ | 1 | 2 | 2 |
| | 236 | 51 | | | Ig kappa chain V-I region BAN | IPI00385555 | LTQ | 1 | 2 | 3 |
| | 237 | 52 | | | Ig kappa chain V-I region Ni | IPI00387106 | LTQ | 0.96 | 1 | 3 |
| | 238 | 53 | | | Ig kappa chain V-II region MIL | IPI00387110 | LTQ | 1 | 4 | 15 |

TABLE 12

| | | | Newly | | | | | Proba- | | |
|---|---|---|---|---|---|---|---|---|---|---|
| | | Sub- | detected | Detected | | | | bility | Number | Total |
| Venn | Total | group | in | in | | | | of | of | number |
| diagram | num- | num- | vitreous | plasma | | IPI accession | | Protein | unique | of |
| location | ber | ber | proteome | proteome | Protein name | number | Method | Prophet | peptides | peptides |
| | 239 | 54 | | | Ig kappa chain V-III region IARC/BL41 | IPI00386131 | LTQ | 0.96 | 1 | 3 |
| | | | | | precursor | | | | | |
| | 240 | 55 | | | Ig kappa chain V-III region NG9 precursor | IPI00387116 | LTQ | 1 | 3 | 9 |
| | | | | | (Fragment) | | | | | |
| | 241 | 56 | | * | IGHA1 PROTEIN | IPI00166866 | MAL | 1 | 1 | 1 |
| | 242 | 57 | | * | IGL@ PROTEIN | IPI00154742 | MAL | 1 | 5 | 9 |
| | 243 | 58 | | | Insulin-like growth factor-binding protein 6 | IPI00029235 | LTQ | 1 | 3 | 3 |
| | | | | | precursor | | | | | |
| | 244 | 59 | | | Insulin-like growth factor-binding protein 7 | IPI00016915 | LTQ | 1 | 6 | 15 |
| | | | | | precursor | | | | | |
| | 245 | 60 | | * | Insulin-like growth factor-binding protein | IPI00020996 | LTQ | 1 | 4 | 8 |
| | | | | | complex acid labile chain precursor | | | | | |
| | 246 | 61 | | | inter-alpha trypsin inhibitor heavy chain | IPI00328829 | LTQ | 1 | 3 | 5 |
| | | | | | precursor 5 isoform 1 | | | | | |
| | 247 | 62 | | | Isoform 1 of Amyloid-like protein 2 precursor | IPI00031030 | LTQ | 1 | 24 | 46 |
| | 248 | 63 | * | * | Isoform 1 of Attractin precursor | IPI00027235 | LTQ | 1 | 16 | 24 |
| | 249 | 64 | * | | Isoform 1 of Cartilage acidic protein 1 | IPI00451624 | LTQ | 1 | 5 | 10 |
| | | | | | precursor | | | | | |
| | 250 | 65 | * | * | Isoform 1 of Contactin-1 precursor | IPI00029751 | LTQ | 1 | 6 | 7 |
| | 251 | 66 | * | * | Isoform 1 of Ectonucleotide | IPI00156171 | LTQ | 1 | 44 | 65 |
| | | | | | pyrophosphatase/phosphodiesterase 2 | | | | | |
| | 252 | 67 | | | Isoform 1 of EGF-containing fibulin-like | IPI00029658 | LTQ | 1 | 6 | 13 |
| | | | | | extracellular matrix protein 1 precursor | | | | | |
| | 253 | 68 | * | * | Isoform 1 of Interleukin-6 receptor subunit beta | IPI00297124 | LTQ | 1 | 3 | 3 |
| | | | | | precursor | | | | | |
| | 254 | 69 | * | * | Isoform 1 of N-acetylmuramoyl-L-alanine | IPI00163207 | LTQ | 1 | 19 | 35 |
| | | | | | amidase precursor | | | | | |
| | 255 | 70 | * | | Isoform 1 of Neuronal cell adhesion molecule | IPI00333776 | LTQ | 1 | 14 | 15 |
| | | | | | precursor | | | | | |
| | 256 | 71 | * | * | Isoform 1 of Pappalysin-2 precursor | IPI00013569 | LTQ | 1 | 16 | 17 |
| | 257 | 72 | * | * | Isoform 1 of Sex hormone-binding globulin | IPI00023019 | LTQ | 1 | 9 | 16 |
| | | | | | precursor | | | | | |
| | 258 | 73 | * | | Isoform 1 of Target of Nesh-SH3 precursor | IPI00440822 | LTQ | 1 | 16 | 26 |
| | 259 | 74 | * | | Isoform 1 of Tenascin precursor | IPI00031008 | LTQ | 1 | 6 | 8 |
| | 260 | 75 | * | | Isoform 1 of Tripeptidyl-peptidase 1 precursor | IPI00298237 | LTQ | 1 | 6 | 11 |
| | 261 | 76 | * | | Isoform 1 of VPS10 domain-containing | IPI00103597 | LTQ | 1 | 3 | 6 |
| | | | | | receptor SorCS1 precursor | | | | | |
| | 262 | 77 | | | Isoform 2 of Apolipoprotein-L1 precursor | IPI00186903 | LTQ | 1 | 6 | 9 |
| | 263 | 78 | * | * | Isoform 2 of Neural cell adhesion molecule L1- | IPI00299059 | LTQ | 1 | 11 | 11 |
| | | | | | like protein precursor | | | | | |
| | 264 | 79 | * | * | Isoform 2 of Reelin precursor | IPI00241562 | LTQ | 1 | 2 | 2 |
| | 265 | 80 | | | Isoform A of Osteopontin precursor | IPI00021000 | LTQ | 1 | 2 | 5 |
| | 266 | 81 | * | | Isoform A of Protein CutA precursor | IPI00034319 | LTQ | 1 | 3 | 5 |
| | 267 | 82 | | | Isoform A3 of Beta crystallin A3 | IPI00010847 | LTQ | 1 | 10 | 16 |
| | 268 | 83 | | * | Isoform APP770 of Amyloid beta A4 protein | IPI00006608 | LTQ | 1 | 13 | 33 |
| | | | | | precursor (Fragment) | | | | | |
| | 269 | 84 | | | Isoform B of Fibulin-1 precursor | IPI00218803 | LTQ | 1 | 4 | 4 |
| | 270 | 85 | | * | Isoform DPI of Desmoplakin | IPI00013933 | LTQ | 1 | 9 | 15 |
| | 271 | 86 | * | * | Isoform N-CAM 120 of Neural cell adhesion | IPI00220737 | LTQ | 1 | 5 | 8 |
| | | | | | molecule 1, 120 kDa isoform precursor | | | | | |
| | 272 | 87 | * | | Isoform Short of Receptor-type tyrosine-protein | IPI00216283 | LTQ | 1 | 5 | 8 |
| | | | | | phosphatase zeta precursor | | | | | |
| | 273 | 88 | * | | Isoform V0 of Versican core protein precursor | IPI00009802 | LTQ | 1 | 9 | 27 |
| | 274 | 89 | * | | Junction plakoglobin | IPI00554711 | LTQ | 1 | 5 | 6 |
| | 275 | 90 | | | K12 keratin [Homo sapiens] | gi\|2497269\|sp\| | LTQ | 1 | 2 | 4 |
| | 276 | 91 | | | keratin 10, type I, epidermal - human | gi\|88041\|pir\|\| | LTQ | 1 | 51 | 424 |
| | | | | | | A31994 | | | | |
| | 277 | 92 | | * | Keratin 6 irs3 | IPI00174775 | LTQ | 1 | 3 | 7 |
| | 278 | 93 | | | Keratin 77 | IPI00376379 | LTQ | 1 | 4 | 24 |
| | 279 | 94 | | | keratin K5, 58K type II, epidermal | gi\|88052\|pir\|\| | LTQ | 1 | 3 | 9 |
| | | | | | (version 2) - human (fragment) | A32568 | | | | |
| | 280 | 95 | | | Keratin, type I cytoskeletal 14 | IPI00384444 | LTQ | 1 | 25 | 61 |
| | 281 | 96 | | * | Keratin, type I cytoskeletal 9 | IPI00019359 | MAL | 1 | 12 | 21 |
| | 282 | 97 | | | KERATIN, TYPE II CYTOSKELETAL 5 | gi\|125105\|sp\|P | LTQ | 1 | 6 | 9 |
| | | | | | (CYTOKERATIN 5) (K5) (CK 5) (58 KD | | | | | |
| | | | | | CYTOKERATIN) | | | | | |
| | 283 | 98 | | | Keratin-78 | IPI00166205 | LTQ | 1 | 3 | 4 |
| | 284 | 99 | * | * | Low-density lipoprotein receptor-related | IPI00020557 | LTQ | 1 | 4 | 4 |
| | | | | | protein 1 precursor | | | | | |
| | 285 | 100 | * | * | Low-density lipoprotein receptor-related | IPI00024292 | LTQ | 1 | 9 | 20 |
| | | | | | protein 2 precursor | | | | | |
| | 286 | 101 | | * | Lumican precursor | IPI00020986 | LTQ | 1 | 9 | 40 |
| | 287 | 102 | | * | Lysozyme C precursor | IPI00019038 | LTQ | 1 | 3 | 5 |
| | 288 | 103 | | | Metalloproteinase inhibitor 2 precursor | IPI00027166 | LTQ | 1 | 4 | 5 |
| | 289 | 104 | * | * | Monocyte differentiation antigen CD14 | IPI00029260 | LTQ | 1 | 15 | 29 |
| | | | | | precursor | | | | | |
| | 290 | 105 | | | Myosin-reactive immunoglobulin light chain | IPI00384399 | LTQ | 0.96 | 1 | 1 |
| | | | | | variable region (Fragment) | | | | | |
| | 291 | 106 | * | | N(4)-(beta-N-acetylglucosaminyl)-L- | IPI00026259 | LTQ | 1 | 2 | 3 |
| | | | | | asparaginase [Precursor] | | | | | |
| | 292 | 107 | * | | N-acetyllactosaminide beta-1,3-N- | IPI00009997 | MAL | 1 | 9 | 11 |
| | | | | | acetylglucosaminyltransferase | | | | | |
| | 293 | 108 | * | * | Neuroserpin precursor | IPI00016150 | LTQ | 1 | 6 | 8 |
| | 294 | 109 | | | Opticin precursor | IPI00002678 | MAL | 1 | 11 | 22 |
| | 295 | 110 | * | | Palmitoyl-protein thioesterase 1 precursor | IPI00002412 | LTQ | 1 | 3 | 3 |
| | 296 | 111 | * | * | phosphatidylethanolamine-binding protein 4 | IPI00163563 | LTQ | 1 | 4 | 5 |
| | 297 | 112 | * | * | Prolactin-inducible protein precursor | IPI00022974 | LTQ | 1 | 2 | 2 |
| | 298 | 113 | * | | Protein CREG1 precursor | IPI00021997 | LTQ | 1 | 2 | 2 |

TABLE 13

| | | | Newly | | | | | Proba- | | |
|---|---|---|---|---|---|---|---|---|---|---|
| | | Sub- | detected | Detected | | | | bility | Number | Total |
| Venn | Total | group | in | in | | | | of | of | number |
| diagram | num- | num- | vitreous | plasma | | IPI accession | | Protein | unique | of |
| location | ber | ber | proteome | proteome | Protein name | number | Method | Prophet | peptides | peptides |
| | 299 | 114 | | | Protein FAM3C precursor | IPI00021923 | LTQ | 1 | 6 | 19 |
| | 300 | 115 | * | | Protein OAF homolog | IPI00328703 | MAL | 1 | 3 | 9 |
| | 301 | 116 | * | * | Protein S100-A8 | IPI00007047 | LTQ | 0.94 | 1 | 1 |
| | 302 | 117 | | * | Prothrombin precursor (Fragment) | IPI00019568 | LTQ | 1 | 29 | 74 |
| | 303 | 118 | | | Retinoschisin precursor | IPI00007331 | LTQ | 1 | 9 | 22 |
| | 304 | 119 | * | | Ribonuclease pancreatic precursor | IPI00014048 | LTQ | 0.96 | 1 | 2 |
| | 305 | 120 | * | * | Secretogranin-3 precursor | IPI00292071 | LTQ | 0.96 | 1 | 2 |
| | 306 | 121 | * | * | seizure related 6 homolog | IPI00154734 | LTQ | 1 | 11 | 19 |
| | 307 | 122 | * | | Semaphorin-7A precursor | IPI00025257 | LTQ | 1 | 6 | 9 |
| | 308 | 123 | * | * | similar to hephaestin isoform 1 | IPI00261031 | LTQ | 1 | 4 | 6 |
| | 309 | 124 | * | | similar to Plexin-B2 precursor | IPI00398435 | LTQ | 1 | 7 | 7 |
| | 310 | 125 | * | * | SPARC precursor | IPI00014572 | LTQ | 1 | 5 | 6 |
| | 311 | 126 | * | | SPARC-like protein 1 precursor | IPI00296777 | LTQ | 1 | 5 | 10 |
| | 312 | 127 | | | Spondin-1 precursor | IPI00171473 | LTQ | 1 | 21 | 38 |
| | 313 | 128 | * | * | Tau-tubulin kinase | IPI00217437 | LTQ | 0.99 | 2 | 18 |
| | 314 | 129 | * | | type 1 tumor necrosis factor receptor shedding | IPI00165949 | LTQ | 1 | 3 | 3 |
| | | | | | aminopeptidase regulator isoform a | | | | | |
| | 315 | 130 | | | type I keratin 16 - human | gi\|1363944\|pir | LTQ | 1 | 25 | 55 |
| | 316 | 131 | | * | Type I transmembrane receptor precursor | IPI00018276 | LTQ | 1 | 5 | 5 |
| | 317 | 132 | | | TYPE II CYTOSKELETAL 2 EPIDERMAL | gi\|547754\|sp\|P | LTQ | 1 | 32 | 165 |
| | | | | | (CYTOKERATIN 2E) (K2E) (CK 2E) | | | | | |
| | 318 | 133 | * | | Vasorin precursor | IPI00395488 | LTQ | 1 | 4 | 7 |
| | 319 | 134 | * | | Vesicular integral-membrane protein VIP36 | IPI00009950 | LTQ | 1 | 7 | 8 |
| | | | | | precursor | | | | | |
| | 320 | 135 | | | vitamin D-binding protein precursor | IPI00555812 | MAL | 1 | 16 | 43 |
| | 321 | 136 | | * | Vitamin K-dependent protein C precursor | IPI00021817 | LTQ | 0.96 | 1 | 1 |
| | 322 | 137 | | * | Vitamin K-dependent protein S precursor | IPI00294004 | LTQ | 0.96 | 1 | 2 |
| | 323 | 138 | * | | Wnt inhibitory factor 1 precursor | IPI00001863 | MAL | 1 | 22 | 69 |
| E | 324 | 1 | * | * | 187 kDa protein | IPI00164623 | MAL | 1 | 137 | 865 |
| | 325 | 2 | * | | 26 kDa protein | IPI00480016 | MAL | 1 | 2 | 64 |
| | 326 | 3 | | * | ALB protein | IPI00022434 | MAL | 1 | 36 | 283 |
| | 327 | 4 | | | Alpha crystallin A chain | IPI00021062 | LTQ | 1 | 12 | 41 |
| | 328 | 5 | | * | Alpha-1-acid glycoprotein 1 precursor | IPI00022429 | MAL | 1 | 48 | 509 |
| | 329 | 6 | | * | Alpha-1-acid glycoprotein 2 precursor | IPI00020091 | MAL | 1 | 30 | 186 |
| | 330 | 7 | | | Alpha-1-antitrypsin precursor | IPI00553177 | MAL | 1 | 131 | 2750 |
| | 331 | 8 | | * | Alpha-1B-glycoprotein precursor | IPI00022895 | MAL | 1 | 39 | 192 |
| | 332 | 9 | | * | alpha-2-glycoprotein 1, zinc | IPI00166729 | MAL | 1 | 8 | 135 |
| | 333 | 10 | | * | Alpha-2-HS-glycoprotein precursor | IPI00022431 | MAL | 1 | 20 | 93 |
| | 334 | 11 | | | Alpha-2-macroglobulin precursor | IPI00478003 | MAL | 1 | 204 | 1195 |
| | 335 | 12 | | * | AMBP protein precursor | IPI00022426 | MAL | 1 | 27 | 125 |
| | 336 | 13 | | * | ANTITHROMBIN III VARIANT | IPI00032179 | MAL | 1 | 54 | 376 |
| | 337 | 14 | | * | Apolipoprotein A-I precursor | IPI00021841 | MAL | 1 | 77 | 499 |
| | 338 | 15 | | * | Apolipoprotein A-II precursor | IPI00021854 | MAL | 1 | 12 | 29 |
| | 339 | 16 | | * | Apolipoprotein A-IV precursor | IPI00304273 | MAL | 1 | 21 | 257 |
| | 340 | 17 | | * | APOLIPOPROTEIN D PRECURSOR. | IPI00006662 | LTQ | 1 | 5 | 18 |
| | 341 | 18 | | * | Apolipoprotein E precursor | IPI00021842 | MAL | 1 | 18 | 363 |
| | 342 | 19 | | * | Beta-2-glycoprotein 1 precursor | IPI00298828 | MAL | 1 | 7 | 98 |
| | 343 | 20 | | * | Beta-2-microglobulin precursor | IPI00004656 | MAL | 1 | 5 | 51 |
| | 344 | 21 | * | * | calsyntenin 1 isoform 2 | IPI00007257 | MAL | 1 | 38 | 76 |
| | 345 | 22 | | * | Carbonic anhydrase 1 | IPI00215983 | LTQ | 1 | 15 | 56 |
| | 346 | 23 | | * | Ceruloplasmin precursor | IPI00017601 | MAL | 1 | 147 | 809 |
| | 347 | 24 | | | Chitinase-3-like protein 1 precursor | IPI00002147 | MAL | 1 | 14 | 23 |
| | 348 | 25 | | * | Clusterin precursor | IPI00291262 | MAL | 1 | 75 | 432 |
| | 349 | 26 | | * | Complement C2 precursor (Fragment) | IPI00303963 | LTQ | 1 | 4 | 36 |
| | 350 | 27 | | * | Complement C5 precursor | IPI00032291 | LTQ | 1 | 12 | 14 |
| | 351 | 28 | | | complement component 4B preproprotein | IPI00418163 | MAL | 1 | 10 | 56 |
| | 352 | 29 | | * | Complement component C6 precursor | IPI00009920 | LTQ | 1 | 16 | 28 |
| | 353 | 30 | | * | Complement component C8 gamma chain | IPI00011261 | LTQ | 1 | 7 | 10 |
| | | | | | precursor | | | | | |
| | 354 | 31 | | * | Complement component C9 precursor | IPI00022395 | LTQ | 1 | 20 | 59 |
| | 355 | 32 | | * | Complement factor I precursor | IPI00291867 | MAL | 1 | 7 | 38 |
| | 356 | 33 | | * | Cystatin-C precursor | IPI00032293 | MAL | 1 | 29 | 138 |
| | 357 | 34 | | | cytokeratin 9 [Homo sapiens] | gi\|1082558\|pir | LTQ | 1 | 37 | 324 |
| | 358 | 35 | | * | Glutathione peroxidase 3 precursor | IPI00026199 | MAL | 1 | 28 | 102 |
| | 359 | 36 | | | Hemoglobin subunit alpha | IPI00410714 | MAL | 1 | 15 | 313 |
| | 360 | 37 | | | Hemoglobin subunit beta | IPI00654755 | MAL | 1 | 11 | 248 |

TABLE 14

| | | | Newly | | | | | Proba- | | |
|---|---|---|---|---|---|---|---|---|---|---|
| | | Sub- | detected | Detected | | | | bility | Number | Total |
| Venn | Total | group | in | in | | | | of | of | number |
| diagram | num- | num- | vitreous | plasma | | IPI accession | | Protein | unique | of |
| location | ber | ber | proteome | proteome | Protein name | number | Method | Prophet | peptides | peptides |
| | 361 | 38 | | | Hemoglobin subunit delta | IPIM0473011 | MAL | 1 | 4 | 60 |
| | 362 | 39 | | * | Hemopexin precursor | IPI00022488 | MAL | 1 | 72 | 842 |
| | 363 | 40 | | * | Histidine-rich glycoprotein precursor | IPI00022371 | MAL | 1 | 11 | 28 |
| | 364 | 41 | | | HP protein | IPI00431645 | MAL | 1 | 10 | 16 |
| | 365 | 42 | | | Ig kappa chain V-I region DEE | IPI00387025 | LTQ | 1 | 3 | 54 |
| | 366 | 43 | | | Ig kappa chain V-III region B6 | IPI00387113 | LTQ | 1 | 2 | 7 |
| | 367 | 44 | | | IG KAPPA CHAIN V-III REGION | IPI00784669 | LTQ | 1 | 2 | 43 |
| | | | | | HAH PRECURSOR | | | | | |
| | 368 | 45 | | | IG KAPPA CHAIN V-IV REGION | IPI00387120 | MAL | 0.94 | 4 | 9 |
| | | | | | LEN | | | | | |
| | 369 | 46 | | * | IgGFc-binding protein precursor | IPI00242956 | LTQ | 1 | 32 | 68 |
| | 370 | 47 | | | IGHM PROTEIN | IPI00472610 | LTQ | 1 | 4 | 23 |
| | 371 | 48 | | | IGKC PROTEIN | IPI00430847 | LTQ | 1 | 4 | 36 |
| | 372 | 49 | | | | IPI00746963 | LTQ | 1 | 3 | 37 |
| | 373 | 50 | | | | IPI00807413 | LTQ | 1 | 3 | 39 |
| | 374 | 51 | | | IGKV2-24 protein | IPI00440577 | LTQ | 1 | 1 | 14 |
| | 375 | 52 | | * | Inter-alpha-trypsin inhibitor heavy | IPI00292530 | MAL | 1 | 46 | 155 |
| | | | | | chain H1 precursor | | | | | |
| | 376 | 53 | | * | Inter-alpha-trypsin inhibitor heavy | IPI00305461 | MAL | 1 | 42 | 170 |
| | | | | | chain H2 precursor | | | | | |
| | 377 | 54 | | | Interphotoreceptor retinoid-binding | IPI00022337 | MAL | 1 | 198 | 836 |
| | | | | | protein precursor | | | | | |
| | 378 | 55 | | | Isoform 1 of Alpha-1- | IPI00550991 | MAL | 1 | 68 | 568 |
| | | | | | antichymotrypsin precursor | | | | | |
| | 379 | 56 | | * | Isoform 1 of Complement factor B | IPI00019591 | MAL | 1 | 46 | 165 |
| | | | | | precursor (Fragment) | | | | | |
| | 380 | 57 | | * | Isoform 1 of Complement factor H | IPI00029739 | MAL | 1 | 11 | 27 |
| | | | | | precursor | | | | | |
| | 381 | 58 | | * | Isoform 1 of Fibrinogen alpha chain | IPI00021885 | MAL | 1 | 25 | 96 |
| | | | | | precursor | | | | | |
| | 382 | 59 | | * | Isoform 1 of Gelsolin precursor | IPI00026314 | MAL | 1 | 20 | 177 |
| | 383 | 60 | | * | Isoform 2 of Inter-alpha-trypsin | IPI00218192 | MAL | 1 | 12 | 163 |
| | | | | | inhibitor heavy chain H4 precursor | | | | | |
| | 384 | 61 | | * | Isoform Gamma-B of Fibrinogen | IPI00021891 | MAL | 1 | 29 | 131 |
| | | | | | gamma chain precursor | | | | | |
| | 385 | 62 | | * | Isoform LMW of Kininogen-1 | IPI00215894 | MAL | 1 | 25 | 109 |
| | | | | | precursor | | | | | |
| | 386 | 63 | | | keratin, 67K type II cytoskeletal - | gi\|88054\|pir\|\| | LTQ | 1 | 27 | 230 |
| | | | | | human | A22940 | | | | |
| | 387 | 64 | | | Keratin, type I cytoskeletal 10 | IPI00009865 | MAL | 1 | 7 | 20 |
| | 388 | 65 | | | KERATIN, TYPE I | gi\|547748\|sp\| | LTQ | 1 | 36 | 323 |
| | | | | | CYTOSKELETAL 9 | P35527\|K1CI_HUMAN | | | | |
| | | | | | (CYTOKERATIN 9) (K9) (CK 9) | | | | | |
| | 389 | 66 | | * | Keratin, type II cytoskeletal 1 | IPI00220327 | MAL | 1 | 14 | 75 |
| | 390 | 67 | | * | Leucine-rich alpha-2-glycoprotein | IPI00022417 | MAL | 1 | 18 | 48 |
| | | | | | precursor | | | | | |
| | 391 | 68 | | | Promega trypsin artifact 5K to | Trypa5\|PromTArt5 | LTQ | 1 | 58 | 629 |
| | | | | | R mods (2239.1, 2914)(1987, 2003) | | | | | |
| | 392 | 69 | | * | Pigment epithelium-derived factor | IPI00006114 | MAL | 1 | 62 | 308 |
| | | | | | precursor | | | | | |
| | 393 | 70 | | * | Plasma protease C1 inhibitor | IPI00291866 | MAL | 1 | 17 | 309 |
| | | | | | precursor | | | | | |
| | 394 | 71 | | * | Plasma retinol-binding protein | IPI00022420 | MAL | 1 | 36 | 497 |
| | | | | | precursor | | | | | |
| | 395 | 72 | | * | Plasma serine protease inhibitor | IPI00007221 | LTQ | 1 | 2 | 5 |
| | | | | | precursor | | | | | |
| | 396 | 73 | | * | Plasminogen precursor | IPI00019580 | MAL | 1 | 11 | 103 |
| | 397 | 74 | | * | Prostaglandin-H2 D-isomerase | IPI00013179 | MAL | 1 | 32 | 284 |
| | | | | | precursor | | | | | |
| | 398 | 75 | | * | Serotransferrin precursor | IPI00022463 | MAL | 1 | 234 | 4234 |
| | 399 | 76 | | | SERUM ALBUMIN PRECURSOR | gi\|113576\|sp\|P | LTQ | 1 | 116 | 8808 |
| | 400 | 77 | | * | Serum amyloid A-4 protein precursor | IPI00019399 | LTQ | 1 | 5 | 8 |
| | 401 | 78 | * | * | Serum paraoxonase/arylesterase 1 | IPI00218732 | LTQ | 1 | 11 | 36 |
| | 402 | 79 | | * | Tetranectin precursor | IPI00009028 | LTQ | 1 | 7 | 18 |
| | 403 | 80 | | * | Transthyretin precursor | IPI00022432 | MAL | 1 | 66 | 872 |
| | 404 | 81 | | | Trypsin precursor | gi\|136429\|sp\| | LTQ | 1 | 15 | 1439 |
| | | | | | | P00761\|TRYP_PIG | | | | |
| | 405 | 82 | | | TYPE II CYTOSKELETAL 1 | gi\|1346343\|sp\| | LTQ | 1 | 49 | 408 |
| | | | | | (CYTOKERATIN 1) (K1) | | | | | |
| | | | | | (CK 1) (67 KD CYTOKERATIN) | | | | | |
| | | | | | (HAIR ALPHA PROTEIN) | | | | | |
| | 406 | 83 | | | type II keratin subunit protein | gi\|715326\|pir\|\| | LTQ | 1 | 2 | 7 |
| | | | | | [Homo sapiens] | KRHU2 | | | | |
| | 407 | 84 | | | vitamin D-binding protein precursor | IPI00742696 | LTQ | 1 | 74 | 537 |
| | 408 | 85 | | * | Vitronectin precursor | IPI00298971 | MAL | 1 | 17 | 85 |
| F | 409 | 1 | | | albumin | gi\|229552\|BSA\|\| | LTQ | 1 | 15 | 205 |
| | | | | | | 754920A | | | | |
| | 410 | 2 | | | COMPLEMENT COMPONENT 4A | IPI00643525 | LTQ | 1 | 124 | 431 |
| | 411 | 3 | | | Hypothetical protein | IPI00384938 | MAL | 1 | 2 | 8 |
| | | | | | DKFZp686N02209 | | | | | |
| | 412 | 4 | | | Hypothetical protein LOC649897 | IPI00736860 | LTQ | 1 | 3 | 59 |
| | 413 | 5 | | | IG KAPPA CHAIN V-III REGION | IPI00385253 | LTQ | 1 | 3 | 71 |
| | | | | | CLL PRECURSOR | | | | | |
| | 414 | 6 | | | Ig kappa chain V-III region SIE | IPI00387115 | MAL | 1 | 1 | 2 |
| | 415 | 7 | | | Isoform 2 of Titin | IPI00023283 | LTQ | 1 | 9 | 27 |
| G | 416 | 1 | | | (X90763) HHa5 hair keratin type I | gi\|1668744\|gn [symbol] | LTQ | 1 | 5 | 6 |
| | | | | | intermediate filament [Homo sapiens] | | | | | |
| | 417 | 2 | * | | 106 kDa protein | IPI00293088 | LTQ | 0.95 | 1 | 1 |
| | 418 | 3 | * | | 12 kDa protein | IPI00478441 | LTQ | 0.94 | 1 | 4 |
| | 419 | 4 | * | | 261 KDA PROTEIN | IPI00791343 | LTQ | 1 | 4 | 4 |
| | 420 | 5 | * | * | 31 KDA PROTEIN | IPI00166417 | LTQ | 0.95 | 1 | 1 |
| | 421 | 6 | * | | 53 kDa protein | IPI00020430 | LTQ | 0.96 | 1 | 2 |
| | 422 | 7 | * | * | 72 kDa type IV collagenase precursor | IPI00027780 | LTQ | 0.96 | 1 | 1 |
| | 423 | 8 | * | | Agrin precursor | IPI00374563 | LTQ | 1 | 13 | 13 |

[symbol]

indicates data missing or illegible when filed

TABLE 15

| | | | Newly | | | | | Proba- | | |
|---|---|---|---|---|---|---|---|---|---|---|
| | | Sub- | detected | Detected | | | | bility | Number | Total |
| Venn | Total | group | in | in | | | | of | of | number |
| diagram | num- | num- | vitreous | plasma | | IPI accession | | Protein | unique | of |
| location | ber | ber | proteome | proteome | Protein name | number | Method | Prophet | peptides | peptides |
| | 424 | 9 | | | albumin [Bos primigenius taurus] | gi\|229552\|prf\|\| | LTQ | 0.96 | 1 | 1 |
| | | | | | | 754920A | | | | |
| | 425 | 10 | * | | Alcadein beta | IPI00396423 | LTQ | 0.96 | 1 | 1 |
| | 426 | 11 | * | | Alpha-mannosidase 2 | IPI00003802 | LTQ | 0.96 | 1 | 1 |
| | 427 | 12 | * | | Alpha-N-acetylgalactosaminidase precursor | IPI00414909 | LTQ | 0.96 | 1 | 1 |
| | 428 | 13 | | | Angiogenin precursor | IPI00008554 | LTQ | 0.95 | 1 | 1 |
| | 429 | 14 | | | ANTI-RHD MONOCLONAL T125 | IPI00784817 | LTQ | 0.93 | 1 | 1 |
| | | | | | GAMMA1 HEAVY CHAIN PRECURSOR | | | | | |
| | 430 | 15 | * | | Beta-1,3-N-acetylglucosaminyltransferase | IPI00001793 | LTQ | 0.96 | 1 | 1 |
| | | | | | radical fringe | | | | | |
| | 431 | 16 | | | VATVSLPR 422 ion wrongly assigned z = 3 | Trypa6\|TrypArt6 | LTQ | 0.99 | 2 | 9 |
| | | | | | (1262.8) (llhg are dummy aa's) | | | | | |
| | 432 | 17 | | * | C3 and PZP-like, alpha-2-macroglobulin | IPI00291807 | LTQ | 1 | 5 | 10 |
| | | | | | domain containing 8 | | | | | |
| | 433 | 18 | | * | Cadherin-2 precursor | IPI00290085 | LTQ | 1 | 5 | 7 |
| | 434 | 19 | | | Calsyntenin-2 precursor | IPI00005491 | LTQ | 0.96 | 1 | 1 |
| | 435 | 20 | | * | Carbonic anhydrase-related protein 10 | IPI00024601 | LTQ | 1 | 2 | 2 |
| | 436 | 21 | * | * | Caspase-14 precursor | IPI00013885 | LTQ | 1 | 2 | 2 |
| | 437 | 22 | | | Cathepsin B precursor | IPI00295741 | LTQ | 1 | 3 | 7 |
| | 438 | 23 | * | | CDNA FLJ45402 fis, clone BRHIP3029409, | IPI00384783 | LTQ | 0.92 | 1 | 1 |
| | | | | | moderately similar to Homo sapiens secreted | | | | | |
| | | | | | frizzled-related protein 1 | | | | | |
| | 439 | 24 | * | | Chromogranin A precursor | IPI00290315 | LTQ | 1 | 2 | 5 |
| | 440 | 25 | | * | Coagulation factor V | IPI00022937 | LTQ | 1 | 6 | 6 |
| | 441 | 26 | | | collagen, type VI, alpha 1 precursor | IPI00719088 | LTQ | 1 | 7 | 7 |
| | 442 | 27 | | | complement factor H-related 1 | IPI00167093 | LTQ | 1 | 2 | 4 |
| | 443 | 28 | | | Cystatin-SN precursor | IPI00305477 | LTQ | 1 | 2 | 2 |
| | 444 | 29 | * | | Deoxyribonuclease-2-alpha precursor | IPI00010348 | LTQ | 1 | 2 | 3 |
| | 445 | 30 | * | | DIS3 MITOTIC CONTROL HOMOLOG | IPI00291003 | LTQ | 0.91 | 2 | 2 |
| | | | | | (S. CEREVISIAE)-LIKE | | | | | |
| | 446 | 31 | * | * | EXTL2 protein (Fragment) | IPI00002732 | LTQ | 0.95 | 1 | 1 |
| | 447 | 32 | * | * | Extracellular matrix protein 1 precursor | IPI00003351 | LTQ | 0.95 | 1 | 1 |
| | 448 | 33 | * | * | Full-length cDNA clone CS0DL004YM19 of | IPI00328493 | LTQ | 1 | 4 | 8 |
| | | | | | B cells (Ramos cell line) of Homo sapiens | | | | | |
| | | | | | (Fragment) | | | | | |
| | 449 | 34 | * | * | Glucosidase 2 subunit beta precursor | IPI00026154 | LTQ | 1 | 2 | 2 |
| | 450 | 35 | * | * | Glutaminyl-peptide cyclotransferase | IPI00003919 | LTQ | 1 | 3 | 3 |
| | | | | | precursor | | | | | |
| | 451 | 36 | * | * | Histatin-1 precursor | IPI00012024 | LTQ | 0.92 | 1 | 1 |
| | 452 | 37 | * | | Histone H4 | IPI00453473 | LTQ | 1 | 3 | 3 |
| | 453 | 38 | | | HP protein | IPI00478493 | LTQ | 1 | 32 | 68 |
| | 454 | 39 | | | HYPOTHETICAL PROTEIN | IPI00784807 | LTQ | 1 | 4 | 5 |
| | 455 | 40 | | | HYPOTHETICAL PROTEIN | IPI00426051 | LTQ | 1 | 1 | 1 |
| | | | | | DKFZP686C15213 | | | | | |
| | 456 | 41 | | | HYPOTHETICAL PROTEIN | IPI00784842 | MAL | 1 | 3 | 10 |
| | | | | | DKFZP686G11190 | | | | | |
| | 457 | 42 | | | HYPOTHETICAL PROTEIN | IPI00418153 | LTQ | 1 | 2 | 9 |
| | | | | | DKFZP686I15212 | | | | | |
| | 458 | 43 | | | HYPOTHETICAL PROTEIN | IPI00784998 | MAL | 1 | 3 | 8 |
| | | | | | DKFZP686M24218 | | | | | |
| | 459 | 44 | | | HYPOTHETICAL PROTEIN | IPI00645363 | LTQ | 1 | 3 | 10 |
| | | | | | DKFZP686P15220 | | | | | |
| | 460 | 45 | | | Ig heavy chain V-II region WAH | IPI00382539 | LTQ | 0.93 | 1 | 1 |
| | 461 | 46 | | | IG KAPPA CHAIN V-I REGION SCW | IPI00387101 | LTQ | 0.96 | 2 | 3 |
| | 462 | 47 | | | IG KAPPA CHAIN V-II REGION FR | IPI00387109 | LTQ | 1 | 2 | 2 |
| | 463 | 48 | | | IG KAPPA CHAIN V-III REGION GOL | IPI00385252 | MAL | 1 | 2 | 5 |
| | 464 | 49 | | | IGHG1 PROTEIN | IPI00784810 | LTQ | 0.95 | 1 | 1 |
| | 465 | 50 | | | IGHG4 protein | IPI00550640 | LTQ | 1 | 4 | 16 |
| | 466 | 51 | | | IGKC PROTEIN | IPI00784070 | LTQ | 1 | 1 | 1 |
| | 467 | 52 | | | IGL@ PROTEIN | IPI00719373 | LTQ | 1 | 2 | 27 |
| | 468 | 53 | | | Immunglobulin heavy chain variable region | IPI00745363 | LTQ | 0.96 | 1 | 1 |
| | | | | | (Fragment) | | | | | |
| | 469 | 54 | | * | Isoform 1 of Collagen alpha-1(IX) chain | IPI00294640 | LTQ | 0.96 | 1 | 1 |
| | | | | | precursor | | | | | |
| | 470 | 55 | * | * | Isoform 1 of Contactin-associated protein- | IPI00029343 | LTQ | 0.96 | 1 | 1 |
| | | | | | like 2 precursor | | | | | |
| | 471 | 56 | * | | Isoform 1 of Follistatin-related protein 4 | IPI00477747 | LTQ | 1 | 4 | 5 |
| | | | | | precursor | | | | | |
| | 472 | 57 | * | * | Isoform 1 of L-lactate dehydrogenase | IPI00217966 | LTQ | 1 | 2 | 2 |
| | | | | | A chain | | | | | |
| | 473 | 58 | * | * | Isoform 1 of Neogenin precursor | IPI00023814 | LTQ | 1 | 2 | 2 |
| | 474 | 59 | * | | Isoform 1 of Neural cell adhesion | IPI00027087 | LTQ | 1 | 2 | 3 |
| | | | | | molecule L1 precursor | | | | | |
| | 475 | 60 | * | | Isoform 1 of Neurexin-2-alpha precursor | IPI00007921 | LTQ | 1 | 2 | 2 |
| | 476 | 61 | * | | Isoform 1 of Peptidyl-glycine alpha- | IPI00177543 | LTQ | 0.96 | 1 | 1 |
| | | | | | amidating monooxygenase precursor | | | | | |
| | 477 | 62 | * | * | Isoform 1 of Receptor-type tyrosine-protein | IPI00011642 | LTQ | 0.96 | 1 | 1 |
| | | | | | phosphatase delta precursor | | | | | |
| | 478 | 63 | * | * | Isoform 1 of Sulfhydryl oxidase 1 precursor | IPI00003590 | LTQ | 1 | 8 | 9 |
| | 479 | 64 | * | * | Isoform 1 of Tenascin-R precursor | IPI00160552 | LTQ | 1 | 5 | 10 |
| | 480 | 65 | | * | Isoform 2 of Collagen alpha-1(XVIII) chain | IPI00022822 | LTQ | 1 | 5 | 8 |
| | | | | | precursor | | | | | |
| | 481 | 66 | * | | Isoform 2 of Neurexin-3-alpha precursor | IPI00441515 | LTQ | 1 | 9 | 11 |
| | 482 | 67 | * | | Isoform 2 of Phospholipid transfer protein | IPI00217778 | LTQ | 1 | 7 | 7 |
| | | | | | precursor | | | | | |
| | 483 | 68 | * | | Isoform 2 of Testican-3 precursor | IPI00419590 | LTQ | 1 | 2 | 2 |
| | 484 | 69 | * | | Isoform 2 of Triosephosphate isomerase | IPI00451401 | LTQ | 0.9 | 1 | 1 |

TABLE 16

| | | | Newly | | | | | Proba- | | |
|---|---|---|---|---|---|---|---|---|---|---|
| | | Sub- | detected | Detected | | | | bility | Number | Total |
| Venn | Total | group | in | in | | | | of | of | number |
| diagram | num- | num- | vitreous | plasma | | IPI accession | | Protein | unique | of |
| location | ber | ber | proteome | proteome | Protein name | number | Method | Prophet | peptides | peptides |
| | 485 | 70 | * | | Isoform 4 of Seizure 6-like protein | IPI00157417 | LTQ | 1 | 4 | 4 |
| | | | | | precursor | | | | | |
| | 486 | 71 | | * | Isoform C of Fibulin-1 precursor | IPI00296537 | LTQ | 1 | 3 | 3 |
| | 487 | 72 | * | | Isoform Long of Alpha-mannosidase | IPI00027703 | LTQ | 1 | 3 | 3 |
| | | | | | IIx | | | | | |
| | 488 | 73 | * | | Isoform Long of Iduronate 2-sulfatase | IPI00026104 | LTQ | 1 | 6 | 6 |
| | | | | | precursor | | | | | |
| | 489 | 74 | * | * | Isoform Sap-mu-0 of Proactivator | IPI00012503 | LTQ | 1 | 2 | 3 |
| | | | | | polypeptide precursor | | | | | |
| | 490 | 75 | * | * | ISOFORM XB OF TENASCIN-X | IPI00025276 | LTQ | 0.91 | 2 | 2 |
| | | | | | PRECURSOR | | | | | |
| | 491 | 76 | | | Kappa light chain variable region | IPI00743194 | LTQ | 1 | 2 | 5 |
| | | | | | (Fragment) | | | | | |
| | 492 | 77 | | | keratin, 48K type I microfibrillar, | gi\|71531\|pir\|\| | LTQ | 1 | 7 | 11 |
| | | | | | component 8c-1 - sheep | KRSHL1 | | | | |
| | 493 | 78 | | | KERATIN, GLYCINE/TYROSINE- | gi\|547810\|sp\| | LTQ | 0.96 | 1 | 2 |
| | | | | | RICH OF HAIR | Q02958\|KRHA_SHEEP | | | | |
| | 494 | 79 | | * | Keratin, type I cuticular Ha3-II | IPI00031423 | LTQ | 0.97 | 1 | 1 |
| | 495 | 80 | | | KERATIN, TYPE I | gi\|125090\|sp\| | LTQ | 0.98 | 1 | 1 |
| | | | | | MICROFIBRILLAR 48 KD, | P02534\|K1M1_SHEEP | | | | |
| | | | | | COMPONENT 8C-1 | | | | | |
| | | | | | (LOW-SULFUR KERATIN) | | | | | |
| | 496 | 81 | | | Keratin, type II cytoskeletal 3 | IPI00290857 | LTQ | 0.99 | 3 | 5 |
| | 497 | 82 | | | KERATIN, TYPE II | gi\|547753\|sp\|P | LTQ | 1 | 3 | 3 |
| | | | | | CYTOSKELETAL 4 | | | | | |
| | | | | | (CYTOKERATIN 4) (K4) (CK4) | | | | | |
| | 498 | 83 | | | KERATIN, TYPE II | gi\|125117\|sp\| | LTQ | 1 | 2 | 2 |
| | | | | | MICROFIBRILLAR, | P25691\|K2M3_SHEEP, | | | | |
| | | | | | COMPONENT 5 | gi\| | | | | |
| | | | | | | 346581\|pir\|\|S29094 | | | | |
| | 499 | 84 | | | KERATIN, TYPE II | gi\|125116\|sp\| | LTQ | 1 | 5 | 6 |
| | | | | | MICROFIBRILLAR, | P15241\|K2M2_SHEEP, | | | | |
| | | | | | COMPONENT 7C | gi\| | | | | |
| | | | | | | 89882\|pir\|\|S05408 | | | | |
| | 500 | 85 | * | | Laminin subunit beta-2 precursor | IPI00296922 | LTQ | 0.95 | 1 | 1 |
| | 501 | 86 | * | * | Laminin subunit gamma-1 precursor | IPI00298281 | LTQ | 0.96 | 1 | 1 |
| | 502 | 87 | * | | Latent-transforming growth factor | IPI00292150 | LTQ | 1 | 2 | 2 |
| | | | | | beta-binding protein 2 precursor | | | | | |
| | 503 | 88 | * | | Legumain precursor | IPI00293303 | LTQ | 0.94 | 1 | 1 |
| | 504 | 89 | * | * | L-lactate dehydrogenase B chain | IPI00219217 | LTQ | 0.95 | 1 | 1 |
| | 505 | 90 | * | | Lysosomal protective protein | IPI00021794 | LTQ | 1 | 2 | 2 |
| | | | | | precursor | | | | | |
| | 506 | 91 | * | | Malate dehydrogenase, cytoplasmic | IPI00291005 | LTQ | 0.96 | 1 | 1 |
| | 507 | 92 | | * | Metalloproteinase inhibitor 1 | IPI00032292 | LTQ | 1 | 3 | 4 |
| | | | | | precursor | | | | | |
| | 508 | 93 | | | MYOSIN-REACTIVE | IPI00384407 | LTQ | 1 | 2 | 2 |
| | | | | | IMMUNOGLOBULIN HEAVY | | | | | |
| | | | | | CHAIN VARIABLE REGION | | | | | |
| | | | | | (FRAGMENT) | | | | | |
| | 509 | 94 | | | Myosin-reactive immunoglobulin | IPI00549330 | LTQ | 1 | 3 | 9 |
| | | | | | light chain variable region | | | | | |
| | 510 | 95 | * | | N-acetylglucosamine-6-sulfatase | IPI00012102 | LTQ | 1 | 5 | 5 |
| | | | | | precursor | | | | | |
| | 511 | 96 | * | | Neurocan core protein precursor | IPI00159927 | LTQ | 1 | 2 | 2 |
| | 512 | 97 | * | | Neuronal pentraxin-2 precursor | IPI00026946 | LTQ | 1 | 2 | 3 |
| | 513 | 98 | | | keratin type II, KII-9, hair - sheep | gi\|109048\|pir\|\| | LTQ | 1 | 13 | 18 |
| | | | | | gi\|1308 (X62509) hair | S22025 | | | | |
| | | | | | type II keratin intermediate | | | | | |
| | | | | | filament protein [Ovis aries] | | | | | |
| | 514 | 99 | * | * | Oligodendrocyte-myelin glycoprotein | IPI00295832 | LTQ | 1 | 4 | 8 |
| | | | | | precursor | | | | | |
| | 515 | 100 | * | * | Protein S100-A9 | IPI00027462 | LTQ | 1 | 2 | 2 |
| | 516 | 101 | * | | retbindin | IPI00027765 | LTQ | 1 | 2 | 2 |
| | 517 | 102 | * | * | Retinoic acid receptor responder | IPI00019176 | LTQ | 1 | 4 | 5 |
| | | | | | protein 2 precursor | | | | | |
| | 518 | 103 | * | | Secreted frizzled-related | IPI00027596 | LTQ | 0.96 | 1 | 1 |
| | | | | | protein 2 precursor | | | | | |
| | 519 | 104 | * | | Secreted frizzled-related | IPI00294650 | LTQ | 1 | 9 | 20 |
| | | | | | protein 3 precursor | | | | | |
| | 520 | 105 | | | Serine/threonine-protein phosphatase | IPI00298731 | LTQ | 0.95 | 1 | 2 |
| | | | | | 1 regulatory subunit 10 | | | | | |
| | 521 | 106 | * | | similar to 60S ribosomal protein L23a | IPI00001310 | LTQ | 1 | 2 | 14 |
| | 522 | 107 | | | SIMILAR TO IG KAPPA CHAIN | IPI00784430 | LTQ | 1 | 5 | 10 |
| | | | | | V-IV REGION JI PRECURSOR | | | | | |
| | 523 | 108 | | | similar to serine (or cysteine) | IPI00376007 | LTQ | 0.93 | 1 | 2 |
| | | | | | proteinase inhibitor, clade E | | | | | |
| | | | | | (nexin, plasminogen activator | | | | | |
| | | | | | inhibitor type 1), member 2 | | | | | |
| | 524 | 109 | | | STATHERIN PRECURSOR | IPI00022990 | LTQ | 0.96 | 1 | 1 |
| | 525 | 110 | * | * | TBC1 domain family member 1 | IPI00164610 | LTQ | 0.99 | 2 | 3 |
| | 526 | 111 | * | | Testican-1 precursor | IPI00005292 | LTQ | 1 | 4 | 7 |
| | 527 | 112 | | * | THROMBOSPONDIN-1 | IPI00296099 | LTQ | 0.95 | 2 | 2 |
| | | | | | PRECURSOR | | | | | |
| | 528 | 113 | * | * | transmembrane protein 132A | IPI00301865 | LTQ | 1 | 4 | 5 |
| | 529 | 114 | | | isoform b TRYPSINOGEN | gi\|136425\|sp\| | LTQ | 0.98 | 2 | 6 |
| | | | | | | P00760\|TRYP_BOVIN | | | | |
| | 530 | 115 | * | | Two-pore calcium channel protein 2 | IPI00169371 | LTQ | 0.92 | 1 | 1 |
| | 531 | 116 | * | | V2-7 PROTEIN | IPI00747752 | LTQ | 1 | 6 | 65 |

It has been suggested that the proteome profile of vitreous humor is similar to that of serum [24]. However, some proteins have been reported to be present in vitreous samples, e.g., pigment epithelium-derived factor (PEDF), prostaglandin-D2 synthase, plasma glutathione peroxidase, and interphotoreceptor retinoid-binding protein (IRBP) [24], which were also detected in the present study.

Moreover, 240 vitreous proteins, which have not been reported previously in vitreous, were identified during the present study, these include, hepatocyte growth factor activator, kallistatin precursor, thioredoxin, von Willebrand factor (vWF), Wnt inhibitory factor, chromogranin and secreted frizzled-related protein (see Table 8 to 16). Moreover, some of these identified proteins have also been detected in the human plasma proteome (see Table 8 to 16). The 531 vitreous proteins identified in the present study were compared to the plasma proteome generated by the HUPO PPP consortium (Human Proteome Organization, Plasma Proteome Project), which listed 9,504 plasma proteins. Of the 531 proteins in our vitreous proteome, 304 had not been found in plasma, and of the 240 newly detected vitreous proteins 132 had not been found in plasma.

In particular, the locations A, B, C and G in the Venn diagram (FIG. 7C) represent proteins that were detected only in PDR or the control. 185 proteins were only detected in PDR (A, B, and C in FIG. 7C) whereas 116 proteins were detected only in the control (G in FIG. 7C).

(5) Characterization of Vitreous Proteins Via Gene Ontology Annotation

Identified proteins of Table 8 to 16 were annotated using the upper level of gene ontology (GO slim, level 3). Based on Gene Ontology (GO) annotations, we were able to assign “biological process”, “molecular function” and “cellular component” to each identified protein in the depleted PDR, non-depleted PDR, and control MH samples. For the categories “molecular function” and “cellular component” identified proteins most frequently picked up subcategories of “binding” and “extracellular region”, respectively (data not shown).

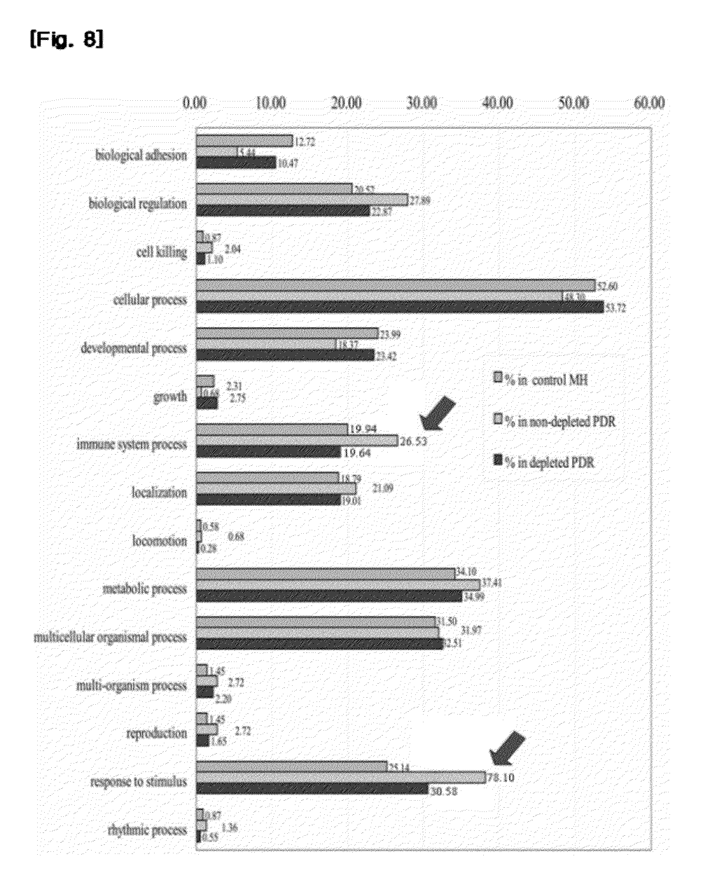

Interestingly, no significant differences were observed between PDR and the control vitreous proteins in terms of patterns of GO annotation, other than the number of proteins assigned to “immune system process” and “response to stimulus” subcategories in the category of “biological process”, which were higher in non-depleted PDR than in control or depleted PDR (FIG. 8). This may indicate that non-depleted PDR contained much more immunoglobulin and complement component species than the other two sample sets because the “immune system process” and “response to stimulus” subcategories comprise more protein products related to the two subcategories. Alternatively, the increase of these two subcategories might be also considered to be the result of increased vascular permeability or breakdown of the blood-retinal barrier in PDR. On the other hand, this increase can also be deducted from the fact that albumin and IgG were substantively removed from the depleted PDR samples.

Consequently, the GO annotation study indicated that there exist diverse kinds of proteins in vitreous, and that they may reflect the physiologic and pathologic changes in retinal disease and vitreoretinal interactions during pathologic conditions. Even though the protein concentrations in PDR and MH vitreous samples differed by 10 fold, protein profiles in the two samples were similar, as inferred from the GO annotation profile category “biological process” (FIG. 8). It is conceivable that the concentrations of most existing vitreous proteins increase with PDR progression, rather than new diverse pathogenic proteins being generated to the extent that they increase protein levels to 10 times that of non-diabetic vitreous proteins.

### 3. Conclusion

In this study, 531 proteins were identified in the vitreous proteome, and 415 and 346 proteins were identified in PDR and control MH vitreous samples, respectively. Of the 531 proteins identified, 240 proteins were identified for the first time during this study. Moreover, 304 of the 531 proteins, including 132 proteins among the newly detected 240 vitreous proteins, were not listed in the HUPO plasma proteome. This list is also the most comprehensive proteome for PDR and normal vitreous samples, and provides fundamental information for those researching vitreoretinal disorders, such as, diabetic retinopathy.

### Example 2

### 1. Materials and Methods

(1) Reagents

β-galactosidase peptides is obtained from Applied Biosystems (USA) and acetonitrile (ACN), formic acid (FA), trifluoro acetic acid (TFA) and most other chemicals such as urea, DTT and IAA are from Sigama (USA). C18 Ziptip for peptide desalting is from Millipore (USA) and trypsin for in-solution digestion of protein is from Promega (Madison, Wis., USA). Vitreous and its corresponding plasma had been collected at Seoul National University Hospital after IRB approval.

(2) Sample Collection

Vitreous samples were collected as described previously. Plasma samples which are corresponding to individual vitreous sample were collected in K₂-EDTA Vacutainer (BD Sciences, USA). After incubating 30 min in room temperature, the centrifugation in 3,000 g during 10 min was followed. Each plasma sample was divided as 50 μl and was kept in −70□.

(3) Concentration Determination

Beforehand, each plasma sample was diluted with 3 volumes of distilled water to be 1/50 diluted in order to reduce pipetting error. BCA assay was carried out using 96 well microplate to determine the concentration of both vitreous and its corresponding plasma. Standard curve was plotted using 5-points of the bovine serum albumin concentration (range: 0.2 μg/μl˜1.0 μg/μl including bank, R²=0.99). After reading the absorbance at 450 nm, each protein concentration was calculated using linear regression method.

(4) Western Blotting

Primary antibody of thyroxine-binding globulin precursor for plasma sample was purchased from Abcam (USA). SDS-PAGE was conducted using 10% gel. Each plasma samples, which are corresponding to the vitreous sample, were applied. Equal amounts of proteins were separated by SDS-PAGE and transferred to PVDF membranes, which were then blocked with 5% BSA (w/v) in TBST 0.1% for 2 hr at room temperature. Membranes were then incubated overnight at 4□ with primary antibodies at a dilution of 1:1000. Blots were visualized using peroxidase-conjugated secondary antibodies and ECL system (Amersham-Pharmacia Biotech, Piscataway, N.J., USA). Band densities were quantified by Phoretix program (Non-linear Dynamics, USA).

(5) Sample Preparation for Mass Spectrometry The same volume of each vitreous (60 μl) was used and 200 μg of each plasma was applied to this analysis. After reducing the volume of each sample using lyophilization, proteins were denatured using 6 M Urea and 10 mM DTT was added to reduce disulfide bonding, followed by alkylation using 55 mM iodoacetamide (IAA). After adding distilled water to dilute the urea concentration, trypsin digestion was carried out (protein: trypsin=50:1). After incubation at 37□ during overnight, 0.1% TFA was added to stop the trypsin digestion. The trypsin-digested peptide mixtures were applied to C18 ZipPlate for desalting, followed by lyophilization. Finally, 10 μl Sol A (98% DW, 2% ACN, 0.1% FA and 0.05% TFA) was added to dissolve peptides for MRM analysis.

(6) Multiple Reaction Monitoring (MRM)

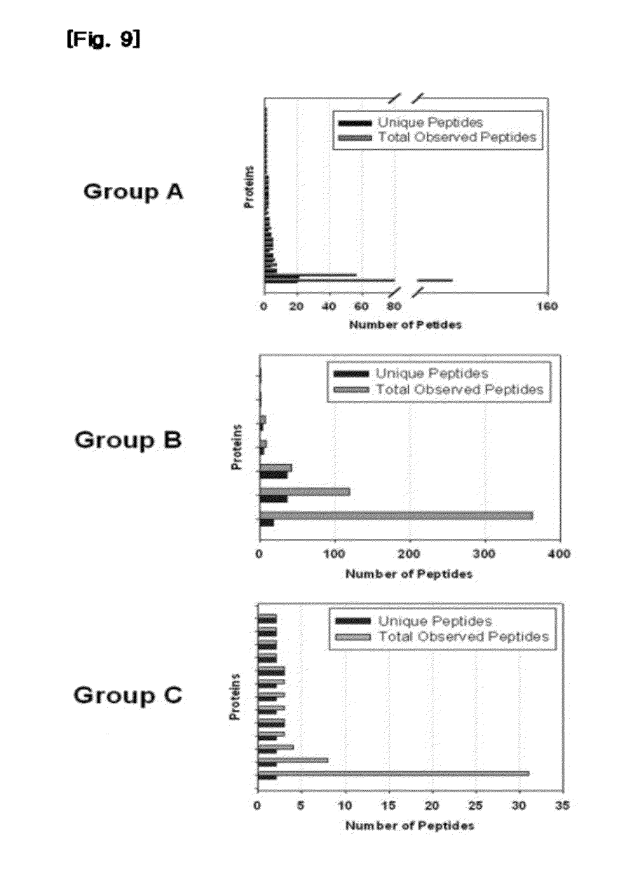

After grouping identified proteins as PDR specific (Groups A, B and C in FIG. 7C), both unique peptides and observed peptides of interesting proteins are accounted. Total number of peptides for each protein that were identified in previous research are counted and plotted in FIG. 9. As another approach, target proteins are selected which show high abundance in any literature. We used 3 different approaches to determine target transitions. The first method is to use LC-MS/MS spectrum from the previous study. The second is to use MIDAS workflow. Thirdly, it is to use the PeptideAtlas database.

Next, the peptide mixtures from vitreous or plasma were applied to mass spectrometry and analyzed with EMS mode followed by four EPI modes. After identification of proteins using ProteinPilot program, the experimental transition are selected from fragment ions in MS/MS spectrum. The MIDAS program can generate the transition candidates from the amino acid sequence. Among these transition candidates, the effective transitions are again confirmed after examining MS/MS spectrum. The PeptideAtlas DB could provide the information of MS/MS spectrum for the interested proteins. Using these MS/MS information, the transitions can be finally determined for the next MRM assay.

With the chosen transitions, MRM assay was performed using 4000 Q-TRAP and nano Tempo MDLC (AppiledBiosystems, USA). Peptide mixtures was separated using C18 column (100 Å, 100 μm ID, 150 mm, Michrome, USA) using Sol A (98% DW, 2% ACN, 0.1% FA and 0.05% TFA) and Sol B (98% ACN, 2% DW, 0.1% FA and 0.05% TFA) with gradient. Flow rate is 400 nl/min as constant at room temperature and exponential gradient elution was performed by increasing the mobile phase composition from 0 to 50% of Sol B over 30 min. The gradient was then ramped to 90% B for 10 min and back to 0% solution B for 20 min to equilibrate the column for the next run. The total LC running time is 60 min. Additionally, to reduce the void volume and obtain sharp transition peak, direct sample injection was carried out from auto sampler to main C18 column using 1 μl sample loop. Ionization was carried out using standard type Nanospary emitter. Spray voltage is 2600 V and declustering potential (DP) was set at 70 V and the time for all transitions was kept at 30 ms. A 4000 Q-TRAP hybrid triple quadrupole linear ion trap mass spectrometer (Applied Biosystems, Foster City, Calif., USA) was interfaced with a nanospray source. Source temperature was set at 160° C., and source voltage was set at 2,600 V. Collision energy (CE) for each transition was based on the results from the preliminary runs and generally was similar to theoretical values calculated from the equations CE=0.044*(m/z)+8 for (M⁺2H⁺) ions and CE=0.030*(m/z)+8 for (M⁺3H⁺) ions.

(7) Data Manipulation and Statistical Analysis

All MRM data were processed using MultiQuant ver. 1.0 (AppliedBiosystems, USA) program for extracting transitions and other calculation. From export of result table, peak area values are extracted and normalized with internal standard transition (530.8/582 from β-galactosidase peptide, of which concentration is 50 fmol). Each normalized peak area of a transition was analyzed to investigate the statistical meaning. The Medicalc, SPSS, and SigmaPlot programs were used for statistical analysis such as pair-wise t-test, ROC curve plotting and interactive plots.

### 2. Results

(1) Characteristics of Vitreous and Corresponding Plasma

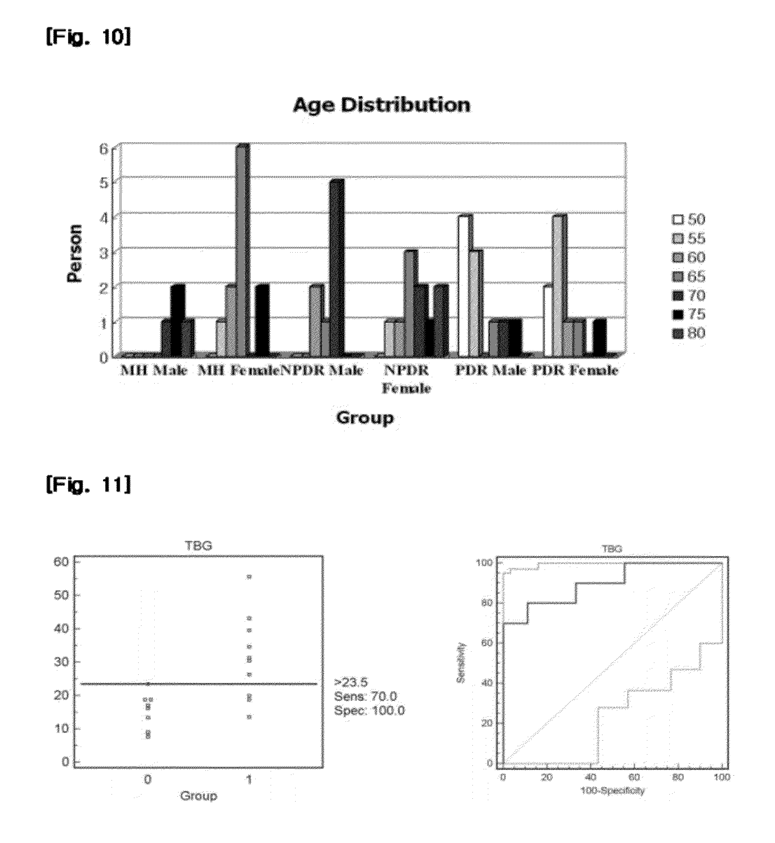

The sample number of MH group was 15 (male: 4, female: 11) and that of NPDR group is 18 (male: 8, NPDR: 10). 18 PDR samples (male: 9, female: 9) was also used to analyze the vitreous/plasma proteome. The age distribution of each group is shown in FIG. 10. The concentration of each vitreous sample group is different from each other. Average concentration of PDR and NPDR is higher than that of MH (Table 17). The concentration of plasma shows the even distribution, which indicates that the variation in vitreous concentration is not related with plasma concentration.

TABLE 17

| | | | Average Protein |
|---|---|---|---|
| Sample set | | Sample | Concentration (μg/μl) |
| (patient numbers) | | Number | (range) |
| Vitreous | MH | 15 | 1.97 (0.40-4.20) |
| | NPDR | 18 | 4.03 (1.11-7.72) |
| | PDR | 18 | 4.54 (2.18-7.52) |
| Plasma | MH | 15 | 65.15 (51.95-82.19) |
| | NPDR | 18 | 81.44 (56.84-106.13) |
| | PDR | 18 | 75.28 (91.06-59.06) |

(2) Transition Selection

The transition representing respective proteins in this study were selected using 3 different ways. The first is MIDAS workflow and the second is utilization of previous data (FIG. 9). MIDAS workflow could provide the theoretical transitions using the protein sequence of which pattern was confirmed by MS/MS experiment. Among several candidate transitions, the best transition, which shows the highest signal, was selected. The second was to use the MS/MS data from other experiments. If the target proteins were identified by other MS experiment, the transition can be selected using its MS/MS spectrum. The third way is the application of peptide database such as Peptide Atlas and GPMDB, which had been identified by other researchers in proteomic fields. These DB provide the informative MS/MS spectrum of peptides that are what we are investigating for.

(3) Standard Curve Determination

The standard curve was determined using β-galactosidase peptide, of which concentration is already known. The range of concentrations was from 100 fmoles to 500 amoles. The correlation factor for linearity is 0.9951, which means that the standard curve of β-galactosidase is reasonable. Using the β-galactosidase standard curve, the relative quantitation for target proteins was extrapolated. To validate the standard curve, the concentration of apolipoprotein A1 was determined using the standard curve of β-galactosidase. The serially diluted plasma was used. The good correlation between the dilution factor and each extrapolated concentrations of apolipoprotein A1 was shown. When the dilution factors increase, the calculated concentrations show the correlation (data not shown).

(4) DR Specific Biomarker In Vitreous

The results of MRM assay for the MH (considered as non-diabetic control), NPDR and PDR vitreous were analyzed with several statistical methods including t-TEST and ROC curve plotting. First, the peak area for each extracted transitions in MRM assay were normalized versus internal standard peak area of β-galactosidase (transitions of 542.3/636.3) which is at 100 fmole concentration. The normalized peak areas of transitions are compared in MH versus PDR and MH versus NPDR. The interactive plots and ROC (receiver operating characteristic) curve, which show the concentration difference for each group, is drawn (MH (non-diabetic control) and PDR, MH (non-diabetic control) and NPDR). Plot for each candidate protein was drawn according to the protein name and transitions.

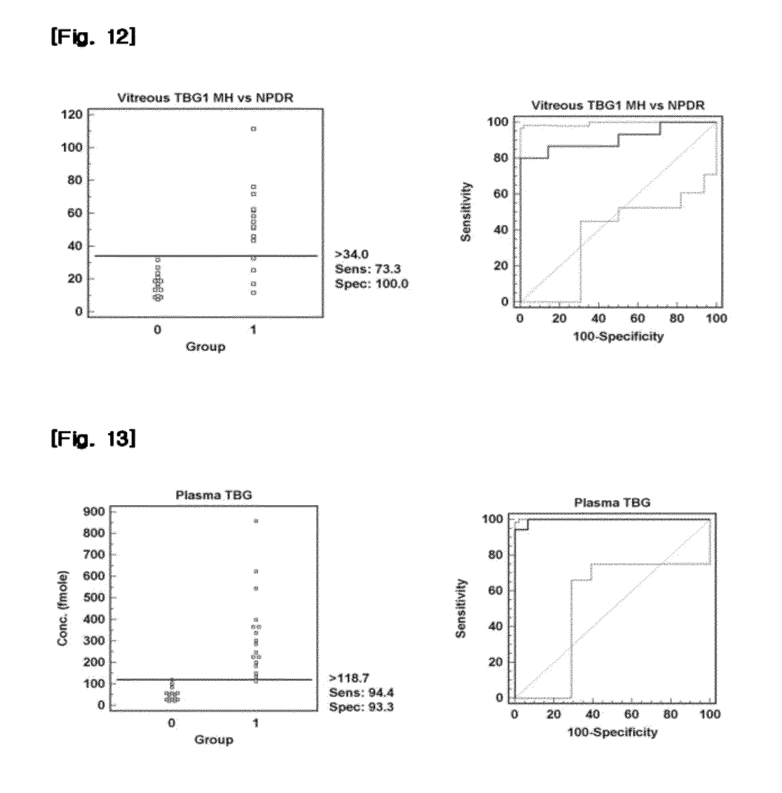

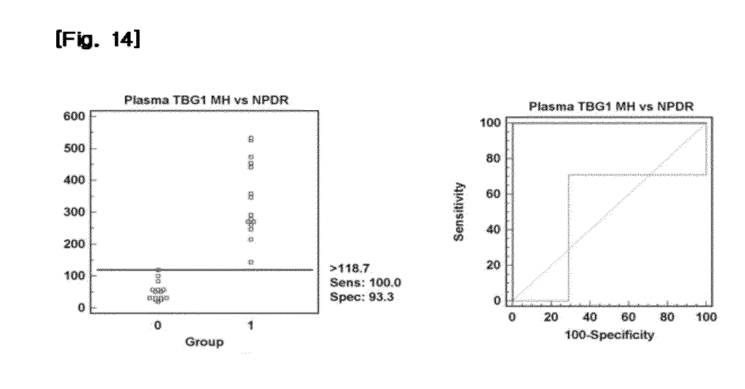

The plots shown in FIG. 11 are the interactive plot and ROC curve of TBG, which is for MH (non-diabetic control) versus PDR in vitreous set. Each interactive plot shows the relatively normalized concentration to 3-galactosidase, sensitivity and specificity. The plots shown in FIG. 12 are the interactive plots and the ROC curves for MH (non-diabetic control) versus NPDR vitreous set. From these two kinds of plots, we could confirm that TBG is clearly differently expressed between two groups. As a result, thyroxine-binding globulin precursor (TBG) shows increase in both PDR and NPDR compared with MH (non-diabetic control) in vitreous sample set.

(6) Diabetic Retinopathy (DR) Specific Biomarker in Plasma

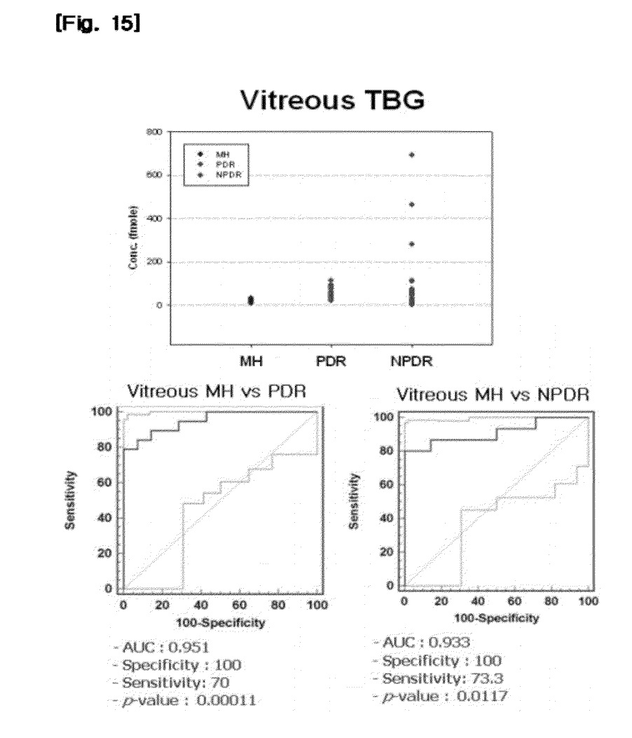

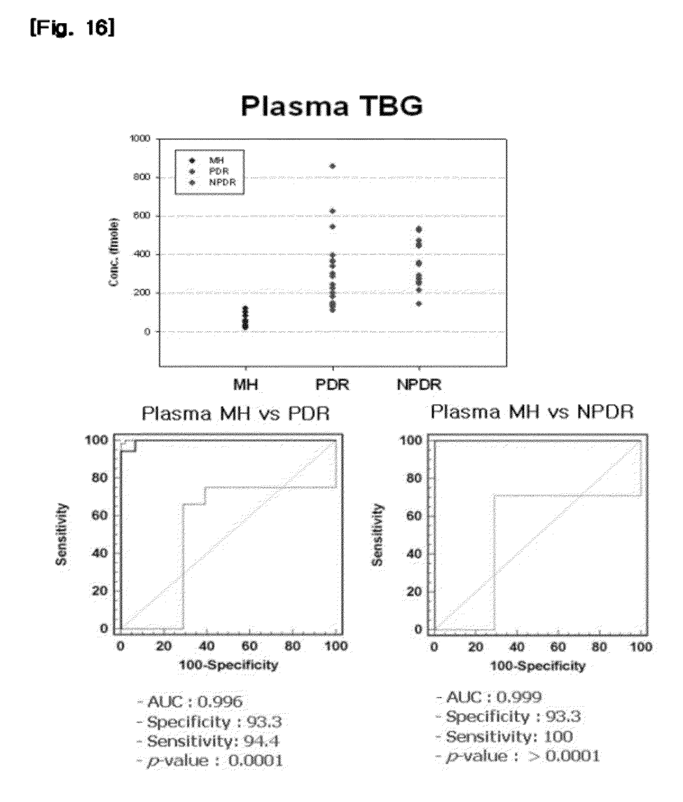

In plasma set, the pattern of thyroxine-binding globulin precursor expression is identical from those for corresponding vitreous samples, where their AUC values were more than 90%. FIG. 13 shows interactive plots of MH versus PDR in plasma sample set. FIG. 14 shows interactive plots and ROC curve of MH versus NPDR in plasma sample set. The vitreous sample set showed excellent AUC value and in plasma sample set, which is the similar case to the PDR versus MH comparison. And thyroxine-binding globulin precursor could be good enough to differentiate NPDR from non-diabetic control plasma, where their AUC values were more than 90%. In summary, based on the interactive plots and ROC curve for both MH versus NPDR and MH versus NPDR in plasma sample set, TBG is biomarkers to differentiate DR plasma from non-diabetic control plasma.

(7) TBG is a Diabetes Mellitus (DM) Biomarker in Both Vitreous and Plasma

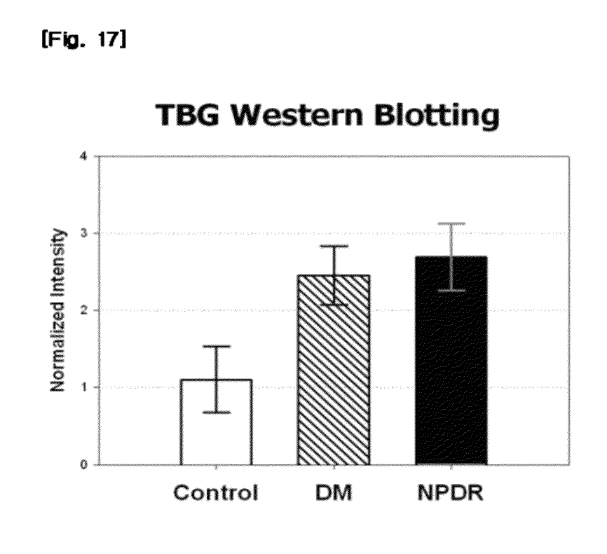

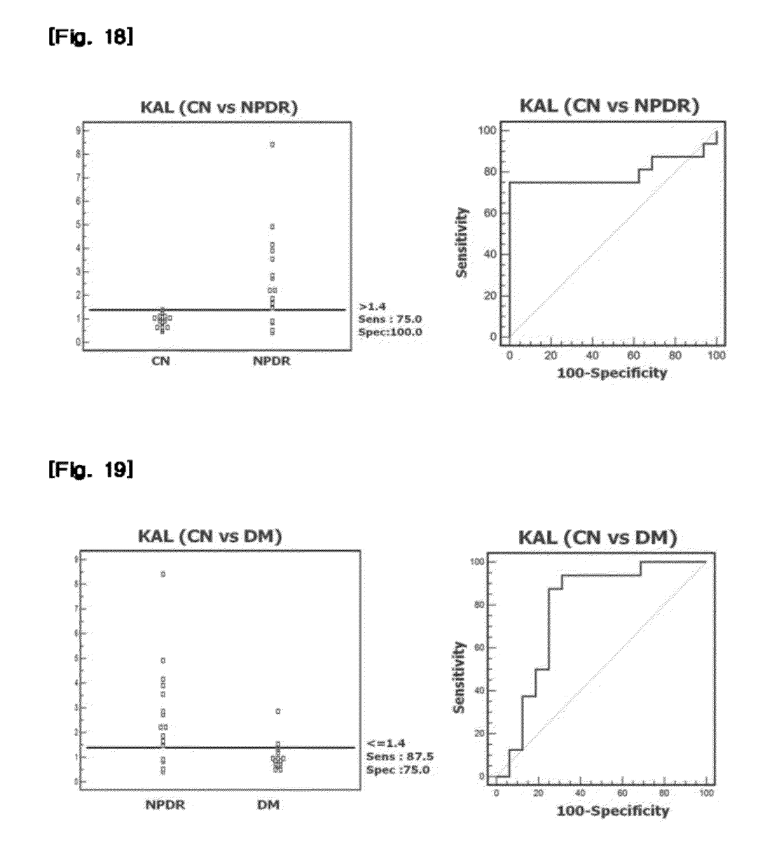

As shown in FIGS. 15 and 16, the levels of thyroxine-binding globulin precursor (TBG) in both vitreous and plasma of PDR and NPDR states are outstandingly higher than in non-diabetic control (MH). It indicates that TBG is a good biomarker which can distinguish both PDR and NPDR from non-diabetic condition. The AUC value of TBG in vitreous and plasma (MH versus PDR and MH versus NPDR) is nearly 1.0 as in below, which indicates its excellent specificity and sensitivity as biomarker.

In order to confirm that TBG is an excellent biomarker, the additional Western-blot assay was performed to validate the effectiveness of TBG. The sample size for the Western blot was 16 healthy normal plasmas, 16 DM plasmas and 16 NPDR plasmas. Each western blot was developed to measure band intensity with densitometry and normalized with total volume of intensity. The averaged intensity of each group was calculated and statistically analyzed.

According to the above Western blot experiment, the significant difference of TBG concentration is observed among disease states (FIG. 17). The healthy control group shows the low level of TBG concentrations in plasma. By contrast, DM and NPDR groups indicated that the levels of TBG are highly increased much more than in that of normal control group. Therefore, it can be concluded that TBG increase in both diabetes and diabetic retinopathy than in healthy normal status. This Western result is corresponding to MRM outcome, which may represent that MRM can determine protein expression properly as efficient as other means. In summary, based on the Western blot data among normal control, DM and NPDR plasma samples, TBG can be a biomarker to distinguish normal control plasma from DM patients including DR plasma.

(8) NPDR Specific Biomarkers in Plasma

Once NPDR occurs, it inevitably develops to PDR. Thus, the value of NPDR biomarkers for DR (including NPDR and PDR) diagnosis should be very high. The discovery of NPDR biomarkers in plasma using MRM assay was performed using the 16 normal control and 16 DM control (DM without DR), and 18 NPDR samples in Table 18.

TABLE 18

| Group | Sex | Sample Number | Age (Median) |
|---|---|---|---|
| Normal control | Male | 8 | 55-69 (60.8) |
| | Female | 8 | 48-77 (60.3) |
| DM control | Male | 8 | 53-70 (60.6) |
| | Female | 8 | 43-70 (58.8) |
| NPDR | Male | 8 | 58-70 (66.0) |
| | Female | 10 | 54-77 (66.5) |

As shown in FIGS. 18 and 19, kallistatin precursor increases in NPDR and decreases in normal states and in DM, which means it can distinguish the NPDR states from the normal and from diabetic states. Therefore, kallistatin precursor can be used for a NPDR specific biomarker.

1. A method for diagnosing diabetic retinopathy or diabetes mellitus, comprising: contacting a molecule specifically binding to the protein as set forth in SEQ ID NO: 69 with a biological sample containing the protein as set forth in SEQ ID NO: 69 as a biomarker; and detecting the binding complex indicative of the diabetic retinopathy or the diabetes mellitus.

2. The method of claim 1, wherein the biological sample is a blood or urine sample.
